(19) United States
(12) Patent Application Publication
(10) Pub. No.: US 2024/0247242 A1
(43) Pub. Date: Jul. 25, 2024

(54) URACIL STABILIZING PROTEINS AND ACTIVE FRAGMENTS AND VARIANTS THEREOF AND METHODS OF USE

(71) Applicant: LIFEEDIT THERAPEUTICS, INC., Morrisville, NC (US)

(72) Inventors: Tyson D. Bowen, Morrisville, NC (US); Alexandra Briner Crawley, Cary, NC (US); Tedd D. Elich, Durham, NC (US)

(73) Assignee: LIFEEDIT THERAPEUTICS, INC., Morrisville, NC (US)

(21) Appl. No.: 18/005,467

(22) PCT Filed: Jul. 15, 2021
(86) PCT No.: PCT/US2021/041809

§ 371 Date: Jan. 13, 2023

Related U.S. Application Data
(60) Provisional application No. 63/052,175, filed on Jul. 15, 2020.

(51) Int. Cl. C12N 9/22; C07K 14/31; C12N 9/78; C12N 15/10

(52) U.S. Cl. CPC C12N 9/22; C07K 14/31; C12N 9/78; C12N 15/102; C07K 2319/09; C12N 2310/20; C12Y 305/04005

(57) ABSTRACT

Compositions and methods comprising uracil stabilizing polypeptides for targeted editing of nucleic acids are provided. Compositions comprise uracil stabilizing polypeptides. Also provided are fusion proteins comprising i) a DNA-binding polypeptide; ii) a deaminase; and iii) a uracil stabilizing polypeptide (USP). The fusion proteins include RNA-guided nucleases fused to deaminases and further fused to a USP, optionally in complex with guide RNAs. Compositions also include nucleic acid molecules encoding the USPs or the fusion proteins. Vectors and host cells comprising the nucleic acid molecules encoding the USPs or the fusion proteins are also provided.

### CROSS-REFERENCE TO RELATED APPLICATIONS

This application is a national stage filing under 35 U.S.C. 371 of International Application no. PCT/US2021/041809 filed Jul. 15, 2021, which was published by the International Bureau in English on Jan. 20, 2022, and which claims priority to U.S. Provisional Application No. 63/052,175, filed Jul. 15, 2020, each of which is incorporated by reference herein in its entirety.

### REFERENCE TO A SEQUENCE LISTING SUBMITTED AS AN ASCII TEXT FILE AT USPTO PATENT CENTER

The application contains a Sequence Listing which is submitted herewith in electronically readable format. The Sequence Listing, created on May 30, 2023, is named L103438 1220US 0155.3 Sequence Listing_ST25.txt and its size is 660,627 bytes. The entire contents of the Sequence Listing are hereby incorporated by reference.

### FIELD OF THE INVENTION

The present invention relates to the field of molecular biology and gene editing.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases (RGNs), such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes through the introduction of a sequence-specific, double-stranded break that is repaired via error-prone non-homologous end-joining (NHEJ) to introduce a mutation at a specific genomic location.

Additionally, RGNs are useful for targeted DNA editing approaches. Targeted editing of nucleic acid sequences, for example targeted cleavage, to allow for introduction of a specific modification into genomic DNA, enables a highly nuanced approach to studying gene function and gene expression. Such targeted editing also may be deployed for targeting genetic diseases in humans or for introducing agronomically beneficial mutations in the genomes of crop plants. The development of genome editing tools provides new approaches to gene editing-based mammalian therapeutics and agrobiotechnology.

### BRIEF SUMMARY OF THE INVENTION

Compositions and methods for modifying a target DNA molecule are provided. The compositions find use in modifying a target DNA molecule of interest. Compositions provided comprise uracil stabilizing polypeptides. Also provided are fusion proteins comprising a DNA-binding polypeptide, a deaminase polypeptide, and a uracil stabilizing polypeptide. Compositions provided also include nucleic acid molecules encoding the uracil stabilizing polypeptides or the fusion proteins, and vectors and host cells comprising the nucleic acid molecules. The methods disclosed herein are drawn to binding a target sequence of interest within a target DNA molecule of interest and modifying the target DNA molecule of interest.

### DETAILED DESCRIPTION

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

This disclosure provides uracil stabilizing polypeptides (USPs), which stabilize uracil residues in a DNA molecule, and nucleic acid molecules encoding the same.

Targeted nucleobase editing, also referred to as base editing, was developed by Komor et al. in 2016 using a cytosine deaminase (rAPOBEC1) operably linked to a modified RNA guided nuclease (SpCas9) (Nature 533: 420-424). In the system described by Komor et al., the guide RNA guides the rAPOBEC1-Cas9 fusion protein to the target DNA sequence, where the rAPOBEC1 deaminates a target cytosine (C) to a uracil (U), which has the base-pairing properties of thymine (T). Using this system, targeted C>T mutations could be introduced into a DNA molecule.

A major drawback for base editing using the rAPOBEC1-Cas9 fusion in vivo was that cellular Uracil DNA Glycosylase (UDG) recognized the U:G heteroduplex DNA and catalyzed the removal of uracil from the DNA to leave an abasic site, thereby initiating base-excision repair with a reversion of the U:G pair to a C:G pair as the most common outcome, although indel (insertion or deletion) formation was also observed. By incorporating a Uracil DNA Glycosylase Inhibitor (UGI) onto the rAPOBEC1-Cas9 fusion protein, the uracil stayed present long enough for replication to occur and introduce the desired C>T mutation.

The present invention finds that by stabilizing the uracil created by the deaminated cytosine, the creation of the abasic site can be prevented and the desired C>T mutation is more likely to be introduced. This was achieved by the identification of Uracil Stabilizing Proteins (also referred to as Uracil Stabilizing Polypeptides, or USPs).

In some embodiments, the USP is provided as part of a fusion protein that comprises a DNA-binding polypeptide, a deaminase polypeptide, and a uracil stabilizing polypeptide. In some embodiments, the DNA-binding polypeptide is or is derived from a meganuclease, zinc finger fusion protein, or TALEN. In some embodiments, the DNA-binding polypeptide is an RNA-guided nuclease, such as a Cas9 polypeptide, that binds to a guide RNA (also referred to as gRNA), which, in turn, binds a target nucleic acid sequence via strand hybridization. In other embodiments, the USP is provided alone.

In some embodiments, the deaminase polypeptide may be a deaminase domain that can deaminate a nucleobase, such as, for example, cytidine. The deamination of a nucleobase by a deaminase can lead to a point mutation at the respective residue, which is referred to herein as “nucleic acid editing”, or “base editing”. Fusion proteins comprising an RNA-guided nuclease (RGN) polypeptide and a deaminase polypeptide can thus be used for the targeted editing of nucleic acid sequences.

Such fusion proteins are useful for targeted editing of DNA in vitro, e.g., for the generation of mutant cells. These mutant cells may be in plants or animals. Such fusion proteins may also be useful for the introduction of targeted mutations, e.g., for the correction of genetic defects in mammalian cells ex vivo, e.g., in cells obtained from a subject that are subsequently re-introduced into the same or another subject; and for the introduction of targeted mutations in vivo, e.g., the correction of genetic defects or the introduction of deactivating mutations in disease-associated genes in a mammalian subject. Such fusion proteins may also be useful for the introduction of targeted mutations in plant cells, e.g., for the introduction of beneficial or agronomically valuable traits or alleles.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### II. Uracil Stabilizing Proteins

Novel uracil-stabilizing polypeptides (USPs) are presently disclosed and set forth as SEQ ID NOs: 1-16. The USPs described herein are useful in applications where stabilizing a uracil in a DNA molecule is desired.

As used herein, the terms “uracil stabilizing protein,” “uracil stabilizing polypeptide,” and “USPs” refer to a polypeptide having uracil stabilizing activity. As used herein, the term “uracil stabilizing activity” refers to the ability of a molecule (e.g., polypeptide) to increase the mutation rate of at least one cytidine, deoxycytidine, or cytosine to a thymidine, deoxythymidine, or thymine in a nucleic acid molecule by a deaminase compared to the mutation rate by the deaminase in the absence of the molecule (e.g., uracil stabilizing polypeptide). Without being bound by a theory or mechanism of action, it is believed that the presently disclosed USPs may function by maintaining the presence of uracil in single-stranded DNA that has been generated through the deamination of a cytidine, deoxycytidine, or cytosine base for a sufficient period of time to allow for replication to occur and introduce the desired C>T mutation. Uracil stabilizing activity may occur through inhibition of uracil DNA glycosylase, the base excision repair pathway, or mis-match repair mechanisms.

In some embodiments, the presently disclosed USPs or active variants or fragments thereof that retain uracil stabilizing activity increase the mutation rate of at least one cytidine, deoxycytidine, or cytosine to a thymidine, deoxythymidine, or thymine in a nucleic acid molecule by a deaminase by at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, at least 96%, at least 97%, at least 98%, at least 99%, at least 100%, at least 150%, at least 200%, or more compared to the mutation rate by a deaminase in the absence of the USP. Conversely, the mutation rate of at least one cytidine, deoxycytidine, or cytosine to any nucleobase other than thymidine, deoxythymidine, or thymine (i.e., guanosine, deoxyguanosine, guanine, adenosine, deoxyadenosine, adenine) in a nucleic acid molecule by a deaminase is reduced by the presently disclosed USPs or active variants or fragments thereof by at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, at least 96%, at least 97%, at least 98%, at least 99%, or more compared to the mutation rate by a deaminase in the absence of the USP. An increase or decrease in the mutation rate of a cytidine, deoxycytidine, or cytosine to another nucleobase can be measured by comparing the rate of mutation of a particular deaminase to a particular nucleobase in the presence or absence of the USP. In those embodiments wherein the deaminase has been targeted to a specific region of a nucleic acid molecule via fusion with a DNA-binding polypeptide, the mutation rate of cytidines, deoxycytidines, or cytosines within or adjacent to the target sequence to which the DNA-binding polypeptide binds can be measured using any method known in the art, including polymerase chain reaction (PCR), restriction fragment length polymorphism (RFLP), or DNA sequencing.

The presently disclosed novel USPs or active variants or fragments thereof that retain uracil stabilizing activity may be introduced into the cell as part of a deaminase-DNA-binding polypeptide fusion, and/or may be co-expressed with a DNA-binding polypeptide-deaminase fusion or with a DNA-binding polypeptide-deaminase-USP fusion, to increase the efficiency of introducing the desired C>T mutation in a target DNA molecule. The presently disclosed USPs retaining uracil stabilizing activity have the amino acid sequence of any of SEQ ID NOs: 1-16 or a variant or fragment thereof. In some embodiments, the USP has an amino acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of any of SEQ ID NOs: 1-16. In particular embodiments, the USP comprises an amino acid sequence having at least 80% sequence identity to any one of SEQ ID NOs: 1, 2, 4, 5, and 7-15. In other embodiments, the USP comprises an amino acid sequence having at least 81% sequence identity to SEQ ID NO: 3 or 16. In still other embodiments, the USP comprises an amino acid sequence having at least 82% sequence identity to SEQ ID NO: 6.

### III. Fusion Proteins

Some aspects of this disclosure provide fusion proteins that comprise a DNA-binding polypeptide and a deaminase polypeptide, and in some embodiments, a USP polypeptide. Such fusion proteins are useful for targeted editing of DNA in vitro, ex vivo, or in vivo.

The term “fusion protein” as used herein refers to a hybrid polypeptide which comprises protein domains from at least two different proteins. A fusion protein may comprise different domains, for example, a DNA-binding domain and a deaminase. In some embodiments, a fusion protein is in a complex with, or is in association with, a nucleic acid, e.g., RNA.

The deaminase polypeptide comprises a deaminase domain that can deaminate a nucleobase, such as, for example, cytidine. The deamination of a nucleobase by a deaminase can lead to a point mutation at the respective residue, which is referred to herein as “nucleic acid editing” or “base editing”. Fusion proteins comprising an RGN polypeptide variant or domain and a deaminase domain can thus be used for the targeted editing of nucleic acid sequences. In some embodiments, a deaminase comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identical to any one of SEQ ID NOs 47, 48 and 76-94. In some embodiments, a deaminase comprises an amino acid sequence at least 80% identical to any one of SEQ ID NOs 47, 48 and 76-94. In some embodiments, a deaminase comprises an amino acid sequence at least 85% identical to any one of SEQ ID NOs 47, 48 and 76-94. In some embodiments, a deaminase comprises an amino acid sequence at least 90% identical to any one of SEQ ID NOs 47, 48 and 76-94. In some embodiments, a deaminase comprises an amino acid sequence at least 95% identical to any one of SEQ ID NOs 47, 48 and 76-94. In other embodiments, a deaminase comprises an amid acid sequence at least 99% identical to any one of SEQ ID NOs 47, 48 and 76-94. In some specific embodiments, a deaminase comprises an amino acid sequence as set forth in any one of SEQ ID NOs 47, 48 and 76-94.

The presently disclosed fusion proteins comprise a DNA-binding polypeptide. As used herein, the term “DNA-binding polypeptide” refers to any polypeptide which is capable of binding to DNA. In certain embodiments, the DNA-binding polypeptide portion of the presently disclosed fusion proteins binds to double-stranded DNA. In particular embodiments, the DNA-binding polypeptide binds to DNA in a sequence-specific manner. As used herein, the terms “sequence-specific” or “sequence-specific manner” refer to the selective interaction with a specific nucleotide sequence.

Two polynucleotide sequences can be considered to be substantially complementary when the two sequences hybridize to each other under stringent conditions. Likewise, a DNA-binding polypeptide is considered to bind to a particular target sequence in a sequence-specific manner if the DNA-binding polypeptide binds to its sequence under stringent conditions. By “stringent conditions” or “stringent hybridization conditions” is intended conditions under which the two polynucleotide sequences (or the polypeptide binds to its specific target sequence) will bind to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3, and the temperature is at least about 30° C. for short sequences (e.g., 10 to 50 nucleotides) and at least about 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1X to 2X SSC (20X SSC =3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5X to 1X SSC at 55 to 60° C. Exemplary high stringency conditions include hybridization in 50% formamide, 1 M NaCl, 1% SDS at 37° C., and a wash in 0.1X SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium. The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

In certain embodiments, the sequence-specific DNA-binding polypeptide is an RNA-guided, DNA-binding polypeptide (RGDBP). As used herein, the terms “RNA-guided, DNA-binding polypeptide” and “RGDBP” refer to polypeptides capable of binding to DNA through the hybridization of an associated RNA molecule with the target DNA sequence.

In some embodiments, the DNA-binding polypeptide of the fusion protein is a nuclease, such as a sequence-specific nuclease. As used herein, the term “nuclease” refers to an enzyme that catalyzes the cleavage of phosphodiester bonds between nucleotides in a nucleic acid molecule. In some embodiments, the DNA-binding polypeptide is an endonuclease, which is capable of cleaving phosphodiester bonds between nucleotides within a nucleic acid molecule, whereas in other embodiments, the DNA-binding polypeptide is an exonuclease that is capable of cleaving the nucleotides at either end (5′ or 3′) of a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 75). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

As used herein, the term “RNA-guided nuclease” or “RGN” refers to an RNA-guided, DNA-binding polypeptide that has nuclease activity. RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence.

Non-limiting examples of RGNs useful in the presently disclosed compositions and methods include those disclosed in Publication Nos. WO 2020/139783, WO 2019/236566, WO 2021/030344, WO/2021/138247, and Application Nos. PCT/US2021/028843 and PCT/US2021/031794, filed Apr. 23, 2021 and May 11, 2021, respectively, each of which is herein incorporated by reference in its entirety. In some embodiments, a presently disclosed fusion protein comprises an RGN comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identical to any one of SEQ ID NOs: 40 and 95-142. In some embodiments, a presently disclosed fusion protein comprises an RGN having an amino acid sequence at least 80% identical to any one of SEQ ID NOs: 40 and 95-142. In some embodiments, a presently disclosed fusion protein comprises an RGN having an amino acid sequence at least 85% identical to any one of SEQ ID NOs: 40 and 95-142. In some embodiments, a presently disclosed fusion protein comprises an RGN having an amino acid sequence at least 90% identical to any one of SEQ ID NOs: 40 and 95-142. In some embodiments, a presently disclosed fusion protein comprises an RGN having an amino acid sequence at least 95% identical to any one of SEQ ID NOs: 40 and 95-142. In other embodiments, a presently disclosed fusion protein comprises an RGN having an amid acid sequence at least 99% identical to any one of SEQ ID NOs: 40 and 95-142. In some specific embodiments, a presently disclosed fusion protein comprises an RGN having an amino acid sequence as set forth in any one of SEQ ID NOs: 40 and 95-142.

According to the present invention, an RGN protein that has been mutated to become nuclease-inactive or “dead”, such as for example dCas9, is herein referred to as an RNA-guided, DNA-binding polypeptide. One exemplary suitable nuclease-inactive Cas9 domain is the D10A/H840A Cas9 domain mutant (see, e.g., Qi et al., Cell. 2013; 152(5): 1173-83, the entire contents of which are incorporated herein by reference). Additionally, suitable nuclease-inactive Cas9 domains of other known RNA guided nucleases (RGNs) can be determined (for example, a nuclease-inactive variant of the RGN APG08290.1 disclosed in U.S. Patent Publication No. 2019/0367949, the entire contents of which are incorporated herein by reference herein).

The term “RGN polypeptide” encompasses RGN polypeptides that only cleave a single strand of a target nucleotide sequence, which is referred to herein as a nickase. Such RGNs have a single functioning nuclease domain. RGN nickases can be naturally-occurring nickases or can be RGN proteins that naturally cleave both strands of a double-stranded nucleic acid molecule that has been mutated within additional nuclease domains such that the nuclease activity of these mutated domains is reduced or eliminated, to become a nickase. In some embodiments, the nickase RGN of the fusion protein comprises a DlOA mutation (for example nAPG07433.1 (SEQ ID NO: 41)) which renders the RGN capable of cleaving only the non-base edited, target strand (the strand which comprises the PAM and is base paired to a gRNA) of a nucleic acid duplex. In some embodiments, the nickase RGN of the fusion protein comprises a DlOA mutation or an equivalent mutation thereof in any one of SEQ ID NOs: 40 and 95-142. In some embodiments, the nickase RGN of the fusion protein comprises a H840A mutation, which renders the RGN capable of cleaving only the base-edited, non-target strand (the strand which does not comprise the PAM and is not base paired to a gRNA) of a nucleic acid duplex. A nickase RGN comprising an H840A mutation, or an equivalent mutation, has an inactivated HNH domain. A nickase RGN comprising a DlOA mutation, or an equivalent mutation, has an inactivated RuvC domain. The deaminase acts on the non-target strand. A nickase comprising a DlOA mutation, or an equivalent mutation, has an inactive RuvC nuclease domain and is not able to cleave the non-targeted strand of the DNA, i.e., the strand where base editing is desired.

Other additional exemplary suitable nuclease inactive Cas9 domains include, but are not limited to, D10A/D839A/H840A, and D10A/D839A/H840A/N863A mutant domains (See, e.g., Mali et al., Nature Biotechnology. 2013; 31(9): 833-838, the entire contents of which are incorporated herein by reference). Additional suitable RGN proteins mutated to be nickases will be apparent to those of skill in the art based on this disclosure and knowledge in the field (such as for example the RGNs disclosed in PCT Publication No. WO 2019/236566) and are within the scope of this disclosure.

In some embodiments the RGN nickase retaining nickase activity comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to nAPG07433.1 (SEQ ID NO: 41).

Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety. RNA-guided nucleases (RGNs) allow for the targeted manipulation of a single site within a genome and are useful in the context of gene targeting for therapeutic and research applications.

In a variety of organisms, including mammals, RNA-guided nucleases have been used for genome engineering by stimulating either non-homologous end joining or homologous recombination. RGNs include CRISPR-Cas proteins, which are RNA-guided nucleases directed to the target sequence by a guide RNA (gRNA) as part of a Clustered Regularly Interspaced Short Palindromic Repeats (CRISPR) RNA-guided nuclease system, or active variants or fragments thereof.

Some aspects of this disclosure provide fusion proteins that comprise an RNA-guided DNA-binding polypeptide, a deaminase polypeptide, and a USP. In some embodiments, the RNA-guided DNA-binding polypeptide is an RNA-guided nuclease. In further embodiments, the RNA-guided nuclease is a naturally-occurring CRISPR-Cas protein or an active variant or fragment thereof. CRISPR-Cas systems are classified into Class I or Class II systems. Class II systems comprise a single effector nuclease and include Types II, V, and VI. Each class is subdivided into types (Types I, II, III, IV, V, VI), with some types further divided into subtypes (e.g., Type II-A, Type II-B, Type II-C, Type V-A, Type V-B).

In certain embodiments, the CRISPR-Cas protein is a naturally-occurring Type II CRISPR-Cas protein or an active variant or fragment thereof As used herein, the term “Type II CRISPR-Cas protein,” “Type II CRISPR-Cas effector protein,” or “Cas9” refers to a CRISPR-Cas effector protein that requires a trans-activating RNA (tracrRNA) and comprises two nuclease domains (RuvC and HNH), each of which is responsible for cleaving a single strand of a double-stranded DNA molecule.

In other embodiments, the CRISPR-Cas protein is a naturally-occurring Type V CRISPR-Cas protein or an active variant or fragment thereof As used herein, the term “Type V CRISPR-Cas protein,” “Type V CRISPR-Cas effector protein,” or “Cas12” refers to a CRISPR-Cas effector protein that cleaves dsDNA and comprises a single RuvC nuclease domain or a split-RuvC nuclease domain and lacks an HNH domain (Zetsche et al 2015, Cell doi:10.1016/j.ce11.2015.09.038; Shmakov et al 2017, Nat Rev Microbiol doi:10.1038/nrmicro.2016.184; Yan et al 2018, Science doi:10.1126/science.aav7271; Harrington et al 2018, Science doi:10.1126/science.aav4294). It is to be noted that Cas12a is also referred to as Cpfl, and does not require a tracrRNA, although other Type V CRISPR-Cas proteins, such as Cas12b, do require a tracrRNA. Most Type V effectors can also target ssDNA (single-stranded DNA), often without a PAM requirement (Zetsche et al 2015; Yan et al 2018; Harrington et al 2018). The term “Type V CRISPR-Cas protein” encompasses the unique RGNs comprising split RuvC nuclease domains, such as those disclosed in U.S. Provisional Appl. No. 62/955,014 filed Dec. 30, 2019, the contents of which are incorporated by reference in its entirety.

In still other embodiments, the CRISPR-Cas protein is a naturally-occurring Type VI CRISPR-Cas protein or an active variant or fragment thereof As used herein, the term “Type VI CRISPR-Cas protein,” “Type VI CRISPR-Cas effector protein,” or “Cas13” refers to a CRISPR-Cas effector proteins that do not require a tracrRNA and comprise two HEPN domains that cleave RNA.The term “guide RNA” refers to a nucleotide sequence having sufficient complementarity with a target nucleotide sequence to hybridize with the target sequence and direct sequence-specific binding of an associated RGN to the target nucleotide sequence. For CRISPR-Cas RGNs, the respective guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. A guide RNA comprises a CRISPR RNA (crRNA) and in some embodiments, a trans-activating CRISPR RNA (tracrRNA).

A CRISPR RNA comprises a spacer sequence and a CRISPR repeat sequence. The “spacer sequence” is the nucleotide sequence that directly hybridizes with the target nucleotide sequence of interest. The spacer sequence is engineered to be fully or partially complementary with the target sequence of interest. In various embodiments, the spacer sequence can comprise from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is about 10 to about 26 nucleotides in length, or about 12 to about 30 nucleotides in length. In particular embodiments, the spacer sequence is about 30 nucleotides in length.

In some embodiments, the degree of complementarity between a spacer sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

The CRISPR RNA repeat sequence comprises a nucleotide sequence that forms a structure, either on its own or in concert with a hybridized tracrRNA, that is recognized by the RGN molecule. In various embodiments, the CRISPR RNA repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In some embodiments, the guide RNA further comprises a tracrRNA molecule. A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. There are often terminal hairpins at the 3′ end of the tracrRNA that can vary in structure and number, but often comprise a GC-rich Rho-independent transcriptional terminator hairpin followed by a string of Us at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 6 nucleotides to about 30 nucleotides, or more. For example, the region of base pairing between the tracrRNA anti-repeat sequence and the CRISPR repeat sequence can be about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the anti-repeat region of the tracrRNA that is fully or partially complementary to a CRISPR repeat sequence is about 10 nucleotides in length. In some embodiments, the degree of complementarity between a CRISPR repeat sequence and its corresponding tracrRNA anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA can comprise from about 60 nucleotides to more than about 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210 or more nucleotides in length. In particular embodiments, the tracrRNA is about 100 to about 201 nucleotides in length, including about 95, about 96, about 97, about 98, about 99, about 100, about 105, about 106, about 107, about 108, about 109, and about 100 nucleotides in length.

Guide RNAs form a complex with the RNA-guided nucleases to direct the RNA-guided nuclease to bind to a target sequence and introduce a single-stranded or double-stranded break at the target sequence. After the target sequence has been cleaved, the break can be repaired such that the DNA sequence of the target sequence is modified during the repair process. Provided herein are methods for using mutant variants of RNA-guided nucleases, which are either nuclease inactive or nickases, which are linked to deaminases to modify a target sequence in the DNA of host cells. The mutant variants of RNA-guided nucleases in which the nuclease activity is inactivated or significantly reduced may be referred to as RNA-guided, DNA-binding polypeptides, as the polypeptides are capable of binding to, but not necessarily cleaving, a target sequence. RNA-guided nucleases only capable of cleaving a single strand of a double-stranded nucleic acid molecule are referred to herein as nickases.

A target nucleotide sequence is bound by an RGN and hybridizes with the guide RNA associated with the RGN. The target sequence can then be subsequently cleaved by the RGN if the polypeptide possesses nuclease activity, which encompasses activity as a nickase.

The guide RNA can be a single guide RNA or a dual-guide RNA system. A single guide RNA comprises the crRNA and optionally tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and optionally tracrRNA are separated by a linker nucleotide sequence.

In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In particular embodiments, the linker nucleotide sequence of a single guide RNA is at least 4 nucleotides in length.

In certain embodiments, the guide RNA can be introduced into a target cell, organelle, or embryo as an RNA molecule. The guide RNA can be transcribed in vitro or chemically synthesized. In other embodiments, a nucleotide sequence encoding the guide RNA is introduced into the cell, organelle, or embryo. In some of these embodiments, the nucleotide sequence encoding the guide RNA is operably linked to a promoter (e.g., an RNA polymerase III promoter). The promoter can be a native promoter or heterologous to the guide RNA-encoding nucleotide sequence.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided nuclease in vitro or in a cell. The chromosomal sequence targeted by the RGN can be a nuclear, plastid or mitochondrial chromosomal sequence. In some embodiments, the target nucleotide sequence is unique in the target genome.

In some embodiments, the target nucleotide sequence is adjacent to a protospacer adjacent motif (PAM). A PAM is generally within about 1 to about 10 nucleotides from the target nucleotide sequence, including about 1, about 2, about 3, about 4, about 5, about 6, about 7, about 8, about 9, or about 10 nucleotides from the target nucleotide sequence. The PAM can be 5′ or 3′ of the target sequence. In some embodiments, the PAM is 3′ of the target sequence. Generally, the PAM is a consensus sequence of about 2-6 nucleotides, but in particular embodiments, can be 1, 2, 3, 4, 5, 6, 7, 8, 9, or more nucleotides in length. Upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1^{st} and 2^{nd}, and 3^{rd}, 3^{rd} and 4^{th}, 4^{th} and 5^{th}, 5^{th} and 6^{th}, 7^{th} and 8^{th}, or 8^{th} and 9^{th} nucleotides from the PAM in either the 5′ or 3′ direction. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (−) strand of the polynucleotide.

RGNs can be used to deliver a fused polypeptide, polynucleotide, or small molecule payload to a particular genomic location. In some embodiments, a nuclease-inactive or a nickase RGN is operably linked to a deaminase and also to a USP of the invention.

As used herein, the term “deaminase” or “deaminase polypeptide” refers to a polypeptide that catalyzes a deamination reaction. The deaminase may be a naturally-occurring deaminase enzyme or an active fragment or variant thereof. In some embodiments, the deaminase is a cytidine deaminase, catalyzing the hydrolytic deamination of cytidine or deoxycytidine to uracil or deoxyuracil, respectively. Cytidine deaminases may work on either DNA or RNA, and typically operate on single-stranded nucleic acid molecules. In preferred embodiments, an RGN which has nickase activity on the target strand nicks the target strand, while the complementary, non-target strand is modified by the deaminase. Cellular DNA-repair machinery may repair the nicked, target strand using the modified non-target strand as a template, thereby introducing a mutation in the DNA.

In some embodiments, the cytidine deaminase is an apolipoprotein B mRNA-editing complex (APOBEC) family deaminase. In some of these embodiments, the deaminase is an APOBEC1 family deaminase. In some embodiments, the cytidine deaminase is an activation-induced cytidine deaminase (AID). In some embodiments, the deaminase is an ACF1/ASE deaminase. In certain embodiments, the deaminase is an adenosine deaminase. In some of these embodiments, the deaminase is an ADAT family deaminase. Additional suitable deaminase enzymes or domains will be apparent to the skilled artisan based on this disclosure.

One exemplary suitable type of deaminase polypeptides are cytidine deaminases, for example, of the APOBEC family. The apolipoprotein B mRNA editing complex (APOBEC) family of cytosine deaminase enzymes encompasses eleven proteins that serve to initiate mutagenesis in a controlled and beneficial manner (Conticello et al., 2008. Genome Biology, 9(6): 229). One family member, activation-induced cytidine deaminase (AID), is responsible for the maturation of antibodies by converting cytosines in ssDNA to uracils in a transcription-dependent, strand-biased fashion (Reynaud et al., 2003. Nature Immunology, 4(7): 631-638). The apolipoprotein B editing complex 3 (APOBEC3) enzyme provides protection to human cells against a certain HIV-1 strain via the deamination of cytosines in reverse-transcribed viral ssDNA (Bhagwat et al., 2004. DNA Repair (Amst), 3(1): 85-9). These proteins all require a Zn²⁺-coordinating motif (HisX- Glu-X₂₃₋₂₆-Pro-Cys-X₂₋₄-Cys) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (wherein W is A or T and R is A or G) for hAID, to TTC for hAPOBEC3F (Navaratnam et al., 2006. Intl J Hematol 83(3): 195-200). A recent crystal structure of the catalytic domain of APOBEC3G revealed a secondary structure comprised of a five-stranded β-sheet core flanked by six a-helices, which is believed to be conserved across the entire family (Holden et al., 2008. Nature 456(7218): 121-124). The active center loops have been shown to be responsible for both ssDNA binding and in determining “hotspot” identity (Chelico et al., 2009. JBiol Chem 284(41): 27761-27765). Overexpression of these enzymes has been linked to genomic instability and cancer, thus highlighting the importance of sequence-specific targeting (Pham et al., 2005. Biochem 44(8): 2703-2715). In some embodiments, the deaminase polypeptide may be a deaminase polypeptide that can deaminate a cytidine to yield a uracil. The deamination of a nucleobase by a deaminase can lead to a point mutation at the respective residue, thereby modifying the DNA molecule. This act of modification is also referred to herein as nucleic acid editing, or base editing. Fusion proteins comprising a Cas9 variant or domain, a deaminase domain, and a USP domain can thus be used for the targeted editing of nucleic acid sequences.

In some embodiments, a nuclease inactive RGN or nickase RGN fused to a deaminase and an USP of the invention can be targeted to a particular location of a nucleic acid molecule (i.e., target nucleic acid molecule), which in some embodiments is a particular genomic locus, to alter the expression of a desired sequence. In some embodiments, the binding of a fusion protein to a target sequence results in deamination a nucleotide base, resulting in conversion from one nucleotide base to another. In some embodiments, the binding of this fusion protein to a target sequence results in deamination of a nucleotide base adjacent to the target sequence. The nucleotide base adjacent to the target sequence that is deaminated and mutated using the presently disclosed compositions and methods may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence (bound by the gRNA) within the target nucleic acid molecule. Some aspects of this disclosure provide fusion proteins comprising (i) a nuclease-inactive or nickase RGN polypeptide; (ii) a deaminase polypeptide; and (iii) a uracil stabilizing polypeptide.

The instant disclosure provides fusion proteins of various configurations. In some embodiments, the deaminase polypeptide is fused to the N-terminus of the RGN polypeptide. In some embodiments, the deaminase polypeptide is fused to the C-terminus of the RGN polypeptide.

In some embodiments, the USP domain, deaminase domain, and RNA-guided, DNA-binding polypeptide are fused to each other via a linker. Various linker lengths and flexibilities between the three functional domains of the fusion protein can be employed (e.g., ranging from very flexible linkers of the form (GGGGS)ₙ and (G)ₙ to more rigid linkers of the form (EAAAK)ₙ and (XP)ₙ in order to achieve the optimal length for deaminase activity for the specific applications. The term “linker,” as used herein, refers to a chemical group or a molecule linking two molecules or moieties, e.g., a binding domain and a cleavage domain of a nuclease. In some embodiments, a linker joins an RNA guided nuclease and a deaminase. In some embodiments, a linker joins a dCas9 and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, the linker comprises a (GGGGS)ₙ, a (G)ₙ an (EAAAK)ₙ, or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof Additional suitable linker motifs and linker configurations will be apparent to those of skill in the art. In some embodiments, suitable linker motifs and configurations include those described in Chen et al., 2013 (Adv Drug Deliv Rev. 65(10):1357-69, the entire contents of which are incorporated herein by reference). Additional suitable linker sequences will be apparent to those of skill in the art.

In some embodiments, the general architecture of exemplary fusion proteins provided herein comprises the structure: [NH₂]-[deaminase]-[RGN polypeptide]-[USP]-[COOH]; [NH₂]-[USP]-[deaminase]-[RGN polypeptide]-[COOH]; [NH₂]-[USP]-[RGN polypeptide]-[deaminase]-[COOH]; [NH 2 ]-[RGN polypeptide]-[deaminase]-[USP]-[COON]; [NH 2 ]-[RGN polypeptide]-[USP polypeptide]-[deaminase polypeptide]-[COON]; or [NH₂]-[deaminase polypeptide]-[USP polypeptide]-[RGN polypeptide]-[COON], wherein NH₂ is the N-terminus of the fusion protein, and COOH is the C-terminus of the fusion protein.

Some aspects of this disclosure provide deaminase-RGN-USP fusion proteins, deaminase-nuclease inactive RGN-USP fusion proteins and deaminase-nickase RGN-USP fusion proteins, with increased C-*T nucleobase editing efficiency as compared to a similar fusion protein that does not comprise a USP domain.

In some embodiments, the fusion protein comprises the structure: [NH₂]-[deaminase]-[nuclease-inactive RGN]-[USP]-[COOH]; [NH₂]-[deaminase polypeptide]-[USP]-[nuclease-inactive RGN]-[COOH]; [NH₂]-[USP]-[deaminase]-[nuclease-inactive RGN]-[COOH]; [NH₂]-[USP]-[nuclease-inactive RGN]-[deaminase]-[COON]; [NH₂]-[nuclease-inactive RGN]-[deaminase]-[USP]-[COON]; or [NH₂]-[nuclease-inactive RGN][USP]-[deaminase]-[COOH]. It should be understood that “nuclease-inactive RGN” represents any RGN, including any CRISPR-Cas protein, which has been mutated to be nuclease-inactive. It should also be understood that “USP” represents one or more USP polypeptides.

In other embodiments, the fusion protein comprises the structure: [NH₂]-[deaminase]-[RGN nickase]-[USP]-[COON]; [NH₂]-[deaminase]-[USP]-[RGN nickase]-[COON]; [NH₂]-[USP]-[deaminase]-[RGN nickase]-[COON]; [NH₂]-[USP]-[RGN nickase]-[deaminase]-[COON]; [NH₂]-[RGN nickase]-[deaminase]-[USP]-[COOH]; or [NH₂]-[RGN nickase]-[USP]-[ deaminase]-[COON]. It should be understood that “RGN nickase” represents any RGN, including any CRISPR-Cas protein, which has been mutated to be active as a nickase. It should also be understood that “USP” represents one or more USP polypeptides.

In some embodiments, the fusion protein comprises a cytidine deaminase having at least 80% sequence identity to any one of SEQ ID NOs: 47, 48 and 76-94, an RGN (or nickase thereof) having at least 80% sequence identity to any one of SEQ ID NOs: 40, 41, 95-142, and a USP having at least 80% sequence identity to any one of SEQ ID NOs: 1-16.

In some embodiments, the fusion protein comprises a cytidine deaminase having at least 85% sequence identity to any one of SEQ ID NOs: 47, 48, and 76-94, an RGN (or nickase thereof) having at least 85% sequence identity to any one of SEQ ID NOs: 40, 41, and 95-142, and a USP having at least 85% sequence identity to any one of SEQ ID NOs: 1-16.

In some embodiments, the fusion protein comprises a cytidine deaminase having at least 90% sequence identity to any one of SEQ ID NOs: 47, 48, and 76-94, an RGN (or nickase thereof) having at least 90% sequence identity to any one of SEQ ID NOs: 40, 41, and 95-142, and a USP having at least 90% sequence identity to any one of SEQ ID NOs: 1-16.

In some embodiments, the fusion protein comprises a cytidine deaminase having at least 95% sequence identity to any one of SEQ ID NOs: 47, 48, and 76-94, an RGN (or nickase thereof) having at least 95% sequence identity to any one of SEQ ID NOs: 40, 41, and 95-142, and a USP having at least 95% sequence identity to any one of SEQ ID NOs: 1-16.

In some embodiments, the fusion protein comprises a cytidine deaminase having the amino acid sequence set forth in any one of SEQ ID NOs: 47, 48, and 76-94, an RGN (or nickase thereof) having the amino acid sequence set forth in any one of SEQ ID NOs: 40, 41 and 95-142, and a USP having the amino acid sequence set forth in any one of SEQ ID NOs: 1-16.

In some embodiments, the “−” used in the general architecture above indicates the presence of an optional linker sequence. In some embodiments, the fusion proteins provided herein do not comprise a linker sequence. In some embodiments, at least one of the optional linker sequences are present.

Other exemplary features that may be present are localization sequences, such as nuclear localization sequences, cytoplasmic localization sequences, export sequences, such as nuclear export sequences, or other localization sequences, as well as sequence tags that are useful for solubilization, purification or detection of the fusion proteins. Suitable localization signal sequences and sequences of protein tags that are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin- tags, S-tags, Softags (e.g., Softag 1, Softag 3), streptags, biotin ligase tags, FlAsH tags, V5 tags, and SBP-tags. Additional suitable sequences will be apparent to those of skill in the art.

In certain embodiments, the presently disclosed fusion proteins comprise at least one cell-penetrating domain that facilitates cellular uptake of the fusion protein. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

In some embodiments, USPs or fusion proteins provided herein further comprise a nuclear localization sequence (NLS). The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the fusion protein.

In some embodiments, the NLS is fused to the N-terminus of the fusion protein or USP. In some embodiments, the NLS is fused to the C-terminus of the fusion protein or USP. In some embodiments, the NLS is fused to the N-terminus of the USP of the fusion protein. In some embodiments, the NLS is fused to the C-terminus of the USP of the fusion protein. In some embodiments, the NLS is fused to the N-terminus of the RGN polypeptide of the fusion protein. In some embodiments, the NLS is fused to the C-terminus of the RGN polypeptide of the fusion protein. In some embodiments, the NLS is fused to the N-terminus of the deaminase polypeptide of the fusion protein. In some embodiments, the NLS is fused to the C-terminus of the deaminase polypeptide of the fusion protein.In some embodiments, the NLS is fused to the fusion protein or UPS via one or more linkers. In some embodiments, the NLS is fused to the fusion protein or UPS without a linker. In some embodiments, the NLS comprises an amino acid sequence of any one of the NLS sequences provided or referenced herein. In some embodiments, the NLS comprises an amino acid sequence as set forth in SEQ ID NO: 42 or SEQ ID NO: 45.

In some embodiments, fusion proteins as provided herein comprise the full-length sequence of a uracil stabilizing protein, e.g., any one of SEQ ID NO: 1-16. In other embodiments, however, fusion proteins as provided herein do not comprise a full-length sequence of a USP, but only a fragment thereof. For example, in some embodiments, a fusion protein provided herein further comprises an RNA-guided, DNA-binding domain, a deaminase domain, and an active fragment of a USP.

In some embodiments, a fusion protein of the invention comprises an RGN, a deaminase, and a USP, wherein the USP has an amino acid sequence of at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to any of SEQ ID NO: 1-16. Examples of such fusion proteins are described in the Examples section here.

In some embodiments, the fusion protein comprises one USP polypeptide. In some embodiments, the fusion protein comprises at least two USP polypeptides, operably linked either directly or via a linker. In some embodiments, the fusion protein comprises one USP polypeptide, and a second USP polypeptide is co-expressed with the fusion protein.

Another embodiment of the invention is a ribonucleoprotein complex comprising the fusion protein and the guide RNA, either as a single guide or as a dual guide RNA (collectively referred to as gRNA).

### IV. Nucleotides Encoding Uracil Stabilizing Polypeptides, Fusion Proteins, and/or gRNA

The present disclosure provides polynucleotides encoding the presently disclosed uracil stabilizing polypeptides (SEQ ID NOs: 17-32). The present disclosure further provides polynucleotides encoding for fusion proteins which comprise a deaminase and DNA-binding polypeptide, for example a meganuclease, a zinc finger fusion protein, or a TALEN. The present disclosure further provides polynucleotides encoding for fusion proteins which comprise a USP, a deaminase domain, and an RNA-guided, DNA-binding polypeptide. Such RNA-guided, DNA-binding polypeptide may be an RGN or RGN variant. The protein variant may be nuclease-inactive or a nickase. The RGN may be a CRISPR-Cas protein or active variant or fragment thereof. SEQ ID NOs: 40 and 41 are non-limiting examples of an RGN and a nickase RGN variant, respectively. Examples of CRISPR-Cas nucleases are well-known in the art, and similar corresponding mutations can create mutant variants which are also nickases or are nuclease inactive.

An embodiment of the invention provides a polynucleotide encoding a fusion protein which comprises an RGN, a deaminase, and a USP described herein (SEQ ID NO: 1-16, or a variant thereof). In some embodiments, a second polynucleotide encodes the guide RNA required by the RGN for targeting to the nucleotide sequence of interest. In other embodiments, the guide RNA and the fusion protein are encoded by the same polynucleotide.

The use of the term “polynucleotide” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, stem-and-loop structures, and the like.

An embodiment of the invention is a nucleic acid molecule comprising a sequence at least 50%, at least 55%, at least 60%, at least 65%, at least 70%, at least 75%, at least 80%, at least 85%, at least 90%, at least 95%, at least 96%, at least 97%, at least 98%, at least 99% or is 100% identical to any of SEQ ID NOs: 17-32, wherein the nucleic acid molecule encodes a USP having uracil stabilizing activity. The nucleic acid molecule may further comprise a heterologous promoter or terminator. The nucleic acid molecule may encode a fusion protein, where the encoded USP is operably linked to a DNA-binding polypeptide, and/or a deaminase. In some embodiments, the nucleic acid molecule encodes a fusion protein, where the encoded USP is operably linked to an RGN and/or a deaminase

Nucleic acid molecules comprising a polynucleotide which encodes a USP of the invention can be codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Polynucleotides encoding the USPs, fusion proteins, and/or gRNAs described herein can be provided in expression cassettes for in vitro expression or expression in a cell, organelle, embryo, or organism of interest. The cassette will include 5′ and 3′ regulatory sequences operably linked to a polynucleotide encoding a USP and/or a fusion protein comprising a USP, an RNA-guided DNA-binding polypeptide and a deaminase, and/or gRNA provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., region coding for a USP, deaminase, RNA-guided DNA-binding polypeptide, and/or gRNA) is a functional link that allows for expression of the coding region of interest. Operably linked elements may be contiguous or non-contiguous. When used to refer to the joining of two protein coding regions, by operably linked is intended that the coding regions are in the same reading frame. Alternatively, the additional gene(s) or element(s) can be provided on multiple expression cassettes. For example, the nucleotide sequence encoding a presently disclosed uracil stabilizing polypeptide, either alone or as a component of a fusion protein, can be present on one expression cassette, whereas the nucleotide sequence encoding a gRNA can be on a separate expression cassette. Another example may have the nucleotide sequence encoding a presently disclosed USP alone on a first expression cassette, a second expression cassette encoding a fusion protein comprising a USP, and a nucleotide sequence encoding a gRNA on third expression cassette. Such an expression cassette is provided with a plurality of restriction sites and/or recombination sites for insertion of the polynucleotides to be under the transcriptional regulation of the regulatory regions. Expression cassettes which comprise a selectable marker gene may also be present.

The expression cassette will include in the 5′-3′ direction of transcription, a transcriptional (and, in some embodiments, translational) initiation region (i.e., a promoter), a USP-encoding polynucleotide of the invention, and a transcriptional (and in some embodiments, translational) termination region (i.e., termination region) functional in the organism of interest. The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adhl promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axigl promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-specific or tissue-preferred promoters can be utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a USP described herein comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

The nucleic acid sequences encoding the USPs, fusion proteins, and/or gRNAs can be operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein.

In certain embodiments, the polynucleotide encoding the USP, fusion protein, and/or gRNA also can be linked to a polyadenylation signal (e.g., SV40 polyA signal and other signals functional in plants) and/or at least one transcriptional termination sequence. Additionally, the sequence encoding the deaminase or fusion protein also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the USP, fusion protein, and/or gRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

In some embodiments, the expression cassette or vector comprising the sequence encoding a fusion protein comprising an RNA-guided DNA-binding polypeptide, such as an RGN, can further comprise a sequence encoding a gRNA. The sequence(s) encoding the gRNA can be operably linked to at least one transcriptional control sequence for expression of the gRNA in the organism or host cell of interest. For example, the polynucleotide encoding the gRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters.

As indicated, expression constructs comprising nucleotide sequences encoding the USPs, fusion proteins, and/or gRNAs can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. The host cell can be a eukaryotic or prokaryotic cell. In particular embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding a deaminase of the invention. It is recognized that other exogenous or endogenous nucleic acid sequences or DNA fragments may also be incorporated into the host cell. Agrobacterium-and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding a deaminase, fusion protein, and/or gRNA includes retroviral, lentiviral, adenoviral, and adeno-associated viral mediated introduction and expression, as well as the use of Caulimoviruses (e.g., cauliflower mosaic virus), Geminiviruses (e.g., bean golden yellow mosaic virus or maize streak virus), and RNA plant viruses (e.g., tobacco mosaic virus).

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G.W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Alternatively, cells that have been transformed may be introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

The polynucleotides encoding the USPs, fusion proteins, and/or gRNAs can be used to transform any eukaryotic species, including but not limited to animals (e.g., mammals, insects, fish, birds, and reptiles), fungi, amoeba, algae, and yeast. The polynucleotides encoding the USPs, fusion proteins, and/or gRNAs can also be used to transform any prokaryotic species, including but not limited to, archaea and bacteria (e.g., Bacillus spp., Klebsiella spp. Streptomyces spp., Rhizobium spp., Escherichia spp., Pseudomonas spp., Salmonella spp., Shigella spp., Vibrio spp., Yersinia spp., Mycoplasma spp., Agrobacterium spp., and Lactobacillus spp.).

Conventional viral and non-viral based gene transfer methods can be used to introduce nucleic acids in mammalian cells or target tissues. Such methods can be used to administer nucleic acids encoding a fusion protein of the invention and optionally a gRNA to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Non-limiting examples include vectors utilizing Caulimoviruses (e.g., cauliflower mosaic virus), Geminiviruses (e.g., bean golden yellow mosaic virus or maize steak virus), and RNA plant viruses (e.g., tobacco mosaic virus). For a review of gene therapy procedures, see Anderson, Science 256: 808- 813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, Agrobacterium-mediated transformation, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291- 297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In some embodiments, the cell or cell line is prokaryotic. In other embodiments, the cell or cell line is eukaryotic. In further embodiments, the cell or cell line is derived from insect, avian, plant, or fungal species. In some embodiments, the cell or cell line may be mammalian, such as for example human, monkey, mouse, cow, swine, goat, hamster, rat, cat, or dog.

A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TF1, CTLL-2, CIR, Rath, CVI, RPTE, A10, T24, 182, A375, ARH-77, Calul, SW480, SW620, SKOV3, SK-UT, CaCo2, P388D1, SEM-K2, WEHI- 231, HB56, TIB55, lurkat, 145.01, LRMB, Bcl-1, BC-3, IC21, DLD2, Raw264.7, NRK, NRK-52E, MRCS, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr-/-, COR-L23, COR-L23/CPR, COR-L235010, CORL23/ R23, COS-7, COV-434, CML Tl, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalcic7, HL-60, HMEC, HT-29, lurkat, lY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1,

NW-145, OPCN/OPCT cell lines, Peer, PNT-1A/ PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with a fusion protein of the invention and optionally a gRNA, or with a ribonucleoprotein complex of the invention, and modified through the activity of fusion protein or ribonucleoprotein complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is an insect. In further embodiments, the insect is an insect pest, such as a mosquito or tick. In other embodiments, the insect is a plant pest, such as a corn rootworm or a fall armyworm. In some embodiments, the transgenic animal is a bird, such as a chicken, turkey, goose, or duck. In some embodiments, the transgenic animal is a mammal, such as a human, mouse, rat, hamster, monkey, ape, rabbit, swine, cow, horse, goat, sheep, cat, or dog.

### V. Variants and Fragments of Polypeptides and Polynucleotides

The present disclosure provides active variants and fragments of naturally-occurring (i.e., wild-type) uracil stabilizing polypeptides, the amino acid sequence of which are set forth as SEQ ID NO: 1-16, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring USPs, such as those disclosed herein, will retain activity such that if they are part of a fusion protein further comprising a deaminase or a fragment thereof and/or a DNA-binding polypeptide or a fragment thereof, said fusion protein will exhibit increased C→T nucleobase editing efficiency as compared to a similar fusion protein that does not comprise a USP domain.

The term “fragment” refers to a portion of a polynucleotide or polypeptide sequence of the invention. “Fragments” or “biologically active portions” include polynucleotides comprising a sufficient number of contiguous nucleotides to retain the biological activity (i.e., deaminase activity on nucleic acids). “Fragments” or “biologically active portions” include polypeptides comprising a sufficient number of contiguous amino acid residues to retain the biological activity. Fragments of the USPs include those that are shorter than the full-length sequences due to the use of an alternate downstream start site. A biologically active portion of a USP can be a polypeptide that comprises, for example, 10, 20, 30, 40, 50, 60, 70, 80, 90, 100, 110, or more contiguous amino acid residues of any of SEQ ID NOs: 1-16, ora variant thereof Such biologically active portions can be prepared by recombinant techniques and evaluated for activity.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In particular embodiments, the presently disclosed polynucleotides encode a USP comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence of any of SEQ ID NOs: 1-16.

A biologically active variant of a uracil stabilizing polypeptide of the invention may differ by as few as about 1-15 amino acid residues, as few as about 1-10, such as about 6-10, as few as 5, as few as 4, as few as 3, as few as 2, or as few as 1 amino acid residue. In specific embodiments, the polypeptides can comprise an N-terminal or a C-terminal truncation, which can comprise at least a deletion of 5, 10, 15, 20, 25, 30, 35, 40, 45, 50, 55, 60, amino acids or more from either the N or C terminus of the polypeptide.

It is recognized that modifications may be made to the USPs provided herein creating variant proteins and polynucleotides. Changes designed by man may be introduced through the application of site-directed mutagenesis techniques. Alternatively, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the uracil stabilizing polypeptide. Alternatively, modifications may be made that improve the activity of the uracil stabilizing polypeptide.

Variant polynucleotides and proteins also encompass sequences and proteins derived from a mutagenic and recombinogenic procedure such as DNA shuffling. With such a procedure, one or more different USPs disclosed herein (e.g., SEQ ID NO: 1-16) is manipulated to create a new USP possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the USP sequences provided herein and other subsequently identified USP genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. 0. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VI. Antibodies

Antibodies to the USPs, fusion proteins, or ribonucleoproteins comprising the USPs of the present invention, including those having the amino acid sequence set forth as SEQ ID NOs: 1-16 or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of USPs or fusion proteins or ribonucleoproteins comprising USPs described herein. Thus, this disclosure provides kits comprising antibodies that specifically bind to the polypeptides or ribonucleoproteins described herein, including, for example, polypeptides comprising a sequence of at least 85% identity to any of SEQ ID NOs: 1-16.

### VII. Systems and Ribonucleoprotein Complexes for Binding a Target Sequence of Interest and Methods of Making the Same

The present disclosure provides a system which targets to a nucleic acid sequence and modifies a target nucleic acid sequence. In some embodiments, an RNA-guided, DNA-binding polypeptide, such as an RGN, and the gRNA are responsible for targeting the ribonucleoprotein complex to a nucleic acid sequence of interest; the deaminase polypeptide is responsible for modifying the targeted nucleic acid sequence from C>U; the uracil stabilizing polypeptide allows the uracil to persist in the DNA molecule so that the desired DNA repair occurs, thereby introducing the C>T mutation. The guide RNA hybridizes to the target sequence of interest and also forms a complex with the RNA-guided, DNA-binding polypeptide, thereby directing the RNA-guided, DNA-binding polypeptide to bind to the target sequence. The RNA-guided, DNA-binding polypeptide is one domain of a 3-domain fusion protein; the second domain is a deaminase, and the third domain is a USP described herein. In some embodiments, the RNA-guided, DNA-binding polypeptide is an RGN, such as a Cas9. Other examples of RNA-guided, DNA-binding polypeptides include RGNs such as those described in U.S. Patent Application Publication No. 2019/0367949 (herein incorporated in its entirety by reference). In some embodiments, the RNA-guided, DNA-binding polypeptide is a Type II CRISPR-Cas polypeptide, or an active variant or fragment thereof. In some embodiments, the RNA-guided, DNA-binding polypeptide is a Type V CRISPR-Cas polypeptide, or an active variant or fragment thereof. In other embodiments, the RNA-guided, DNA-binding polypeptide is a Type VI CRISPR-Cas polypeptide. In other embodiments, the DNA-binding domain of the fusion protein does not require an RNA guide, such as a zinc finger nuclease, TALEN, or meganuclease polypeptide. In some of these embodiments, the nuclease activity of each has been inactivated. In further embodiments, the RNA-guided, DNA-binding polypeptide comprises an amino acid sequence of an RGN, such as an amino acid sequence having at least 80% sequence identity to APG07433.1 (SEQ ID NO: 40) or an active variant or fragment thereof such as nickase APG07433.1 (SEQ ID NO: 41). In further embodiments, the RNA- guided, DNA-binding polypeptide comprises an amino acid sequence of an RGN, such as an amino acid sequence having at least 85% sequence identity to APG07433.1 (SEQ ID NO: 40) or an active variant or fragment thereof such as nickase APG07433.1 (SEQ ID NO: 41). In further embodiments, the RNA-guided, DNA-binding polypeptide comprises an amino acid sequence of an RGN, such as an amino acid sequence 5 having at least 90% sequence identity to APG07433.1 (SEQ ID NO: 40) or an active variant or fragment thereof such as nickase APG07433.1 (SEQ ID NO: 41). In further embodiments, the RNA-guided, DNA-binding polypeptide comprises an amino acid sequence of an RGN, such as an amino acid sequence having at least 95% sequence identity to APG07433.1 (SEQ ID NO: 40) or an active variant or fragment thereof such as nickase APG07433.1 (SEQ ID NO: 41). In further embodiments, the RNA-guided, DNA-binding 10 polypeptide comprises an amino acid sequence of an RGN, such as APG07433.1 (SEQ ID NO: 40) or an active variant or fragment thereof such as nickase APG07433.1 (SEQ ID NO: 41).

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and a fusion protein 15 comprising a deaminase, a USP of the invention, and an RNA-guided, DNA-binding polypeptide as the protein component. The ribonucleoprotein complex can be purified from a cell or organism that has been transformed with polynucleotides that encode the fusion protein and a guide RNA and cultured under conditions to allow for the expression of the fusion protein and guide RNA. Thus, methods are provided for making a USP, a fusion protein, or a fusion protein ribonucleoprotein complex. Such methods comprise 20 culturing a cell comprising a nucleotide sequence encoding a USP, a fusion protein, and in some embodiments a nucleotide sequence encoding a guide RNA, under conditions in which the USP or fusion protein (and in some embodiments, the guide RNA) is expressed. The USP, fusion protein, or fusion ribonucleoprotein can then be purified from a lysate of the cultured cells.

Methods for purifying a USP, fusion protein, or fusion ribonucleoprotein complex from a lysate of a 25 biological sample are known in the art (e.g., size exclusion and/or affinity chromatography, 2D-PAGE,

HPLC, reversed-phase chromatography, immunoprecipitation). In particular methods, the USP or fusion protein is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, 30 E2, FLAG, HA, nus, Softag 1, Softag 3, Strep, SBP, Glu-Glu, HSV, KT3, S, 51, T7, V5, VSV-G, 6xHis, biotin carboxyl carrier protein (BCCP), and calmodulin. Generally, the tagged USP, fusion protein, or fusion ribonucleoprotein complex is purified using immunoprecipitation or other similar methods known in the art.

An “isolated” or “purified” polypeptide, or biologically active portion thereof, is substantially or 35 essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

### VIII. Methods ofModifying a Target Sequence

The present disclosure provides methods for modifying a target nucleic acid molecule (e.g., target DNA molecule) of interest. The methods include delivering a system comprising at least one guide RNA or a polynucleotide encoding the same, and at least one fusion protein comprising a USP of the invention, a deaminase, and an RNA-guided, DNA-binding polypeptide or a polynucleotide encoding the same to the target sequence or a cell, organelle, or embryo comprising the target sequence. In some of these embodiments, the fusion protein comprises any one of the amino acid sequences of SEQ ID NOs: 1-16, or an active variant or fragment thereof.

In some embodiments, the methods comprise contacting a DNA molecule with (a) a fusion protein comprising a USP, a deaminase, and an RNA-guided, DNA-binding polypeptide, such as for example a nuclease-inactive or a nickase Cas9 domain; and (b) a gRNA targeting the fusion protein of (a) to a target nucleotide sequence of the DNA molecule; wherein the DNA molecule is contacted with the fusion protein and the gRNA in an amount effective and under conditions suitable for the deamination of a nucleotide base.

In some embodiments, the target DNA molecule comprises a sequence associated with a disease or disorder, and wherein the deamination of the nucleotide base results in a sequence that is not associated with a disease or disorder. In some embodiments, the disease or disorder affects animals. In further embodiments, the disease or disorder affects mammals, such as humans, cows, horses, dogs, cats, goats, sheep, swine, monkeys, rats, mice, or hamsters. In some embodiments, the target DNA sequence resides in an allele of a crop plant, wherein the particular allele of the trait of interest results in a plant of lesser agronomic value. The deamination of the nucleotide base results in an allele that improves the trait and increases the agronomic value of the plant.

In some embodiments, the desired mutation comprises a T→C point mutation associated with a disease or disorder, and wherein the deamination of the mutant C base results in a sequence that is not associated with a disease or disorder. In some embodiments, the deamination corrects a point mutation in the sequence associated with the disease or disorder.

In some embodiments, the sequence associated with the disease or disorder encodes a protein, and wherein the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

Thus, the presently disclosed compositions and methods can be used for the treatment of a disease or a disorder associated with a sequence (i.e., the sequence is causal for the disease or disorder or causal for symptoms associated with the disease or disorder) that is mutated in order to treat the disease or disorder or the reduction of symptoms associated with the disease or disorder. As used herein, the term “treat” or “treatment” refers to the administration of a pharmaceutical composition disclosed herein comprising a USP or a fusion protein, to a subject having a disease or disorder. Treatment can be prophylactic by preventing the onset of symptoms associated with a disease or disorder in a subject susceptible to the disease or disorder (e.g., genetically predisposed). Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

A pharmaceutical composition is a composition that is employed to prevent, reduce in intensity, cure or otherwise treat a disease or disorder that comprises an active ingredient (i.e., a USP or fusion protein or nucleic acid molecule encoding the same) and a pharmaceutically acceptable carrier. A pharmaceutically acceptable carrier refers to one or more compatible solid or liquid filler, diluents or encapsulating substances which are suitable for administration to a human or other vertebrate animal. In some embodiments, the pharmaceutical composition comprises a pharmaceutically acceptable carrier that is non-naturally occurring.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. Non-limiting examples include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS). Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc. These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Pharmaceutical compositions comprising the USP or fusion proteins or nucleic acid molecules encoding the same or cells comprising the same can be administered to a subject via any route, such as orally, buccally, parenterally, topically, by inhalation or insufflation (i.e., through the mouth or through the nose), or rectally. Administering can be performed, for example, once, a plurality of times, and/or over one or more extended periods.

An effective amount of a pharmaceutical composition of the invention is any amount that is effective to achieve its purpose (e.g., prevention of or recovery from, including partial recovery, or prevention or slowing of disorder or disease caused by a specific sequence). The effective amount, usually expressed in mg/kg can be determined by routine methods during pre-clinical and clinical trials by those of skill in the art.

In those embodiments wherein the method comprises delivering a polynucleotide encoding a guide RNA and/or a fusion protein, the cell or embryo can then be cultured under conditions in which the guide RNA and/or fusion protein are expressed. In various embodiments, the method comprises contacting a target sequence with a ribonucleoprotein complex comprising a gRNA and a fusion protein (which comprises a USP of the invention, a deaminase, and an RNA-guided DNA-binding polypeptide). In certain embodiments, the method comprises introducing into a cell, organelle, or embryo comprising a target sequence a ribonucleoprotein complex of the invention. The ribonucleoprotein complex of the invention can be one that has been purified from a biological sample, recombinantly produced and subsequently purified, or in vitro-assembled as described herein. In those embodiments wherein the ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled ribonucleoprotein complex of the invention can be introduced into a cell, organelle, or embryo using any method known in the art, including, but not limited to electroporation. Alternatively, a fusion protein comprising a USP of the invention, a deaminase, and a RNA-guided, DNA-binding polypeptide, and a polynucleotide encoding or comprising the guide RNA can be introduced into a cell, organelle, or embryo using any method known in the art (e.g., electroporation).

Upon delivery to or contact with the target sequence or cell, organelle, or embryo comprising the target sequence, the guide RNA directs the fusion protein to bind to the target sequence in a sequence-specific manner. The target sequence can subsequently be modified via the deaminase domain and the USP domain of the fusion protein. In some embodiments, the binding of this fusion protein to a target sequence results in modification of a nucleotide adjacent to the target sequence. The nucleotide base adjacent to the target sequence may be 1, 2, 3, 4, 5, 6, 7, 8, 9, 10, 11, 12, 13, 14, 15, 16, 17, 18, 19, 20, 25, 30, 35, 40, 45, 35 50, 55, 60, 65, 70, 75, 80, 85, 90, 95, or 100 base pairs from the 5′ or 3′ end of the target sequence. A fusion protein comprising a USP of the invention, a deaminase, and a RNA-guided, DNA-binding polypeptide can introduce targeted C>T mutations with greater efficiency compared to a fusion protein which comprises a deaminase and an RNA-guided, DNA-binding polypeptide alone.

Methods to measure binding of the fusion protein to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of a RNA-binding, DNA-guided domain, as part of the fusion protein, complexed with more than one guide RNA. The more than one guide RNA can target different regions of a single gene or can target multiple genes. This multiple targeting enables the deaminase domain of the fusion protein to modify nucleic acids, thereby introducing multiple mutations in the target nucleic acid molecule (e.g., genome) of interest. The USP domain of the fusion protein increases the efficacy of introduction of the desired mutations.

In those embodiments wherein the method involves the use of an RNA-guided nuclease (RGN), such as a nickase RGN (i.e., is only able to cleave a single strand of a double-stranded polynucleotide, for example nAPG07433.1 (SEQ ID NO: 41)), the method can comprise introducing two different RGNs or RGN variants that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide. Alternatively, two different fusion proteins may be provided, where each fusion protein comprises a different RGN with a different PAM recognition sequence, so that a greater diversity of nucleotide sequences may be targeted for mutation.

One of ordinary skill in the art will appreciate that any of the presently disclosed methods can be used to target a single target sequence or multiple target sequences. Thus, methods comprise the use of a fusion protein comprising a single RNA-guided, DNA-binding polypeptide in combination with multiple, distinct guide RNAs, which can target multiple, distinct sequences within a single gene and/or multiple genes. The deaminase domain of the fusion protein would then introduce mutations at each of the targeted sequences. The USP domain of the fusion protein increases the efficacy of introduction of the desired mutations. Also encompassed herein are methods wherein multiple, distinct guide RNAs are introduced in combination with multiple, distinct RNA-guided, DNA binding polypeptides. Such RNA-guided, DNA-binding polypeptides may be multiple RGN or RGN variants. These guide RNAs and guide RNA/fusion protein systems can target multiple, distinct sequences within a single gene and/or multiple genes.

### IX. Cells Comprising a Polynucleotide Genetic Modification

Some embodiments provide methods for using the fusion proteins provided herein. In some embodiments, the fusion protein is used to introduce a point mutation into a target nucleic acid molecule by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein. In some embodiments, the purpose of the methods provide herein is to restore the function of a dysfunctional gene via genome editing. The fusion proteins provided herein can be validated for gene editing-based human therapeutics in vitro, e.g., by correcting a disease associated mutation in human cell culture. It will be understood by the skilled artisan that the fusion proteins provided herein, e.g., the fusion proteins comprising a RNA-guided, DNA-binding domain, a deaminase domain, and a USP of the invention can be used to correct any single point C>T mutation. Deamination of the mutant C to U leads to a correction of the mutation.

In some embodiments, a fusion protein comprising an RNA-guided, DNA-binding domain, a deaminase domain, and a USP of the invention may be used for generating mutations in a targeted gene or targeted region of a gene of interest. In some embodiments, a fusion protein of the invention may be used for saturate mutagenesis of a targeted gene or region of a targeted gene of interest followed by high-throughput forward genetic screening to identify novel mutations and/or phenotypes. In other embodiments, a fusion protein described herein may be used for generating mutations in a targeted genomic location, which may or may not comprise coding DNA sequence. Libraries of cell lines generated by the targeted mutagenesis described above may also be useful for study of gene function or gene expression.

Fusion proteins of the invention may also be used to efficiently generate knock-out (KO) lines, including entire libraries of KO lines, through targeted insertion of nonsense mutations. Fusion proteins comprising a RNA-guided, DNA-binding domain, a deaminase domain, and a USP of the invention can convert three codons (CAA, CAG, and CGA) into STOP codons (TAG, TAA, or TGA) if targeted to the coding DNA strain, and can convert TGG into a STOP codon if targeted to the non-coding DNA strain. Billon et al (2017, Mol Cell 67: 1068-1079; incorporated by reference herein) provide a database of over 3.4 million guide RNAs in eight eukaryotic species useful for generation STOP codons. Additionally, Kuscu et al (2017, Nature Methods 14(7): 710-714; incorporated by reference herein) identified 260,000 unique i-stop sgRNAs in the human genome which can target nearly 17,000 genes to introduce early stop codons. In some embodiments, the KO lines are eukaryotic cells. In other embodiments, the KO lines are prokaryotic cells. In some embodiments, the KO lines generated using a fusion protein of the invention are human cell lines. In other embodiments, the KO lines are mammalian cell lines, for example mouse, rat, monkey, cat, dog, cow, pig, sheep, or horse cell lines. In other embodiments, the KO lines are avian cells. In other embodiments, the KO lines are insect cells. In other embodiments, the KO lines are microbial cells. In still other embodiments, the KO lines are plant cells. In further embodiments, the KO lines are Arabidopsis, soybean, maize, cotton, tomato, potato, or bean cells. In further embodiments, the cell lines are plant seeds.

In some embodiments, a fusion protein provided herein may be useful in therapeutic genome editing. For example, a fusion protein comprising a RNA-guided, DNA-binding domain, a deaminase domain, and a USP of the invention may be used to generate targeted nonsense mutations of PCSK9 (proprotein convertase subtilisin/kexin type 9). PCSK9 is involved in lipoprotein homeostasis, and agents which block PCSK9 can lower low-density lipoprotein particle (LDL) concentrations in the blood. Naturally occurring nonsense variants in PCSK9 in individuals result in substantially reduced blood cholesterol levels and reduced risk in coronary heart disease (Cohen et al. (2006) N Engl J Med 354: 1264-1272). Chadwick et al. (2017, Arteriscler Thromb Vasc Biol 37: 1741-1747; incorporated by reference herein) have found that they can successfully introduce targeted C>T mutations into the PCSK9 gene, thereby reducing PCSK9 protein levels and plasma cholesterol levels in mice. In some embodiments, a similar approach may be taken with a fusion protein of the invention. Further, the skilled artisan will understand that the instantly disclosed fusion proteins can be used to correct other point mutations and mutations associated with other cancers and with diseases other than cancer including other proliferative diseases.

Provided herein are cells and organisms comprising a target nucleic acid molecule of interest that has been modified using a process mediated by a fusion protein, optionally with a gRNA as described herein. In some of these embodiments, the fusion protein comprises a USP comprising an amino acid sequence of any of SEQ ID NOs: 1-16, or an active variant or fragment thereof In some embodiments, the fusion protein comprises a USP comprising an amino acid sequence at least 50%, at least 55%, at least 60%, at least 65%, at least 70%, at least 75%, at least 80%, at least 85%, at least 90%, at least 95%, at least 96%, at least 97%, at least 98%, or at least 99% identical to any of SEQ ID NOs: 1-16. In some embodiments, the fusion protein further comprises a deaminase and a RNA-guided, DNA-binding polypeptide. In further embodiments, the fusion protein comprises a deaminase and an RGN or a variant thereof, such as an amino acid sequence having at least 80% sequence identity to APG07433.1 (SEQ ID NO: 40) or its nickase variant nAPG07433.1 (SEQ ID NO: 41). In further embodiments, the fusion protein comprises a deaminase and an RGN or a variant thereof, such as an amino acid sequence having at least 85% sequence identity to APG07433.1 (SEQ ID NO: 40) or its nickase variant nAPG07433.1 (SEQ ID NO: 41). In further embodiments, the fusion protein comprises a deaminase and an RGN or a variant thereof, such as an amino acid sequence having at least 90% sequence identity to APG07433.1 (SEQ ID NO: 40) or its nickase variant nAPG07433.1 (SEQ ID NO: 41). In further embodiments, the fusion protein comprises a deaminase and an RGN or a variant thereof, such as an amino acid sequence having at least 95% sequence identity to APG07433.1 (SEQ ID NO: 40) or its nickase variant nAPG07433.1 (SEQ ID NO: 41). In further embodiments, the fusion protein comprises a deaminase and an RGN or a variant thereof, such as APG07433.1 (SEQ ID NO: 40) or its nickase variant nAPG07433.1 (SEQ ID NO: 41). In other embodiments, the fusion protein comprises a deaminase and a Cas9 or a variant thereof, such as for example dCas9 or nickase Cas9. In some embodiments, the fusion protein comprises a nuclease-inactive or nickase variant of a Type II CRISPR-Cas polypeptide. In other embodiments, the fusion protein comprises a nuclease-inactive or nickase variant of a Type V CRISPR-Cas polypeptide. In still other embodiments, the fusion protein comprises a nuclease-inactive or nickase variant of a Type VI CRISPR-Cas polypeptide.

The modified cells can be eukaryotic (e.g., mammalian, plant, insect, avian cell) or prokaryotic. Also provided are organelles and embryos comprising at least one nucleotide sequence that has been modified by a process utilizing a fusion protein as described herein. The genetically modified cells, organisms, organelles, and embryos can be heterozygous or homozygous for the modified nucleotide sequence.

The mutation(s) introduced by the deaminase domain of the fusion protein can result in altered expression (up-regulation or down-regulation), inactivation, or the expression of an altered protein product or an integrated sequence. In those instances wherein the mutation(s) results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

In other embodiments, the mutation(s) introduced by the deaminase domain of the fusion protein results in the production of a variant protein product. The expressed variant protein product can have at least one amino acid substitution and/or the addition or deletion of at least one amino acid. The variant protein product can exhibit modified characteristics or activities when compared to the wild-type protein, including but not limited to altered enzymatic activity or substrate specificity.

In yet other embodiments, the mutation(s) introduced by the deaminase domain of the fusion protein can result in an altered expression pattern of a protein. As a non-limiting example, mutation(s) in the regulatory regions controlling the expression of a protein product can result in the overexpression or downregulation of the protein product or an altered tissue or temporal expression pattern.

Some aspects of this disclosure provide kits comprising a fusion protein comprising an RNA-guided, DNA-binding polypeptide, such as an RGN polypeptide, for example a nuclease-inactive Cas9 domain, and a deaminase of the invention, and, optionally, a linker positioned between the Cas9 domain and the deaminase. In addition, in some embodiments, the kit comprises suitable reagents, buffers, and/or instructions for using the fusion protein, e.g., for in vitro or in vivo DNA or RNA editing. In some embodiments, the kit comprises instructions regarding the design and use of suitable gRNAs for targeted editing of a nucleic acid sequence.

### X. Additional Applications of USPs

USPs described herein also possess utility beyond genomic base editing. In general, USPs are useful in applications where stabilizing a uracil nucleobase in a DNA molecule is desired. Through a natural process or by the hand of man, a uracil may be introduced into genomic DNA by DNA damage caused by reactive oxygen species, ionizing radiation, and/or alkylating agents. Studies on the mechanisms of DNA repair, such as the base excision repair (BER) pathway, or studies which measure DNA repair capacity, may use a USP of the invention to inhibit repair of the uracil.

Additionally, USPs described herein may be useful for the treatment of various cancers. For example, fluoropyrimidines including 5-fluorouracil (5-FU) and its deoxyribonucleoside metabolite 5-fluorodeoxyuridine (5-FdU) have been widely used in the treatment of various solid tumors, including colorectal cancer. 5-FdU is active through the inhibition of thymidylate synthase, which consequently introduces uracil and 5-FU incorporation into the genome of the cell. As described above, base repair enzymes such as UDG recognize uracil nucleobases in the genomic DNA and remove them. Yan et al (2016; Oncotarget 7 (37): 59299-59313) found that UDG depleted cells were arrested and displayed sustained DNA damage following 5-FdU treatment, indicating that UDG's actions in removal of uracil and 5-FU played a role in the effectiveness of the 5-FdU treatment of the tumor. Delivery of a USP of the invention in combination with fluoropyrimidines may enhance the effectiveness of this treatment of tumors. Thus, pharmaceutical compositions comprising a presently disclosed USP and a fluoropyrimidine are provided, along with methods of treating a cancer with effective amounts of such pharmaceutical compositions. Fluoropyrimidines are a class of anti-cancer antimetabolites that includes capecitabine, carmofur, doxifluridine, fluorouracil, 5-fluorodeoxyuridine, and tegafur.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Non-limiting embodiments include:

1. An isolated polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity and wherein said polypeptide further comprises a heterologous amino acid sequence.

2. The isolated polypeptide of embodiment 1, wherein the amino acid sequence has at least 85% sequence identity to any one of SEQ ID NOs: 1-16.

3. The isolated polypeptide of embodiment 1, wherein the amino acid sequence has at least 90% sequence identity to any one of SEQ ID NOs: 1-16.

4. The isolated polypeptide of embodiment 1, wherein the amino acid sequence has at least 95% sequence identity to any one of SEQ ID NOs: 1-16.

5. The isolated polypeptide of embodiment 1, wherein the amino acid sequence has 100% sequence identity to any one of SEQ ID NOs: 1-16.

6. The isolated polypeptide of embodiment 1, wherein the polypeptide has the sequence of any one of SEQ ID NOs: 33-39.

7. A pharmaceutical composition comprising a non-naturally occurring pharmaceutically acceptable carrier and a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity.

8. The pharmaceutical composition of embodiment 7, wherein the polypeptide comprises an amino acid sequence having at least 85% sequence identity to any one of SEQ ID NOs: 1-16.

9. The pharmaceutical composition of embodiment 7, wherein the polypeptide comprises an amino acid sequence having at least 90% sequence identity to any one of SEQ ID NOs: 1-16.

10. The pharmaceutical composition of embodiment 7, wherein the polypeptide comprises an amino acid sequence having at least 95% sequence identity to any one of SEQ ID NOs: 1-16.

11. The pharmaceutical composition of embodiment 7, wherein the polypeptide comprises an amino acid sequence having 100% sequence identity to any one of SEQ ID NOs: 1-16.

12. A pharmaceutical composition comprising a non-naturally occurring pharmaceutically acceptable carrier and a nucleic acid molecule comprising a polynucleotide encoding a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
   wherein said polypeptide has uracil stabilizing activity.

13. The pharmaceutical composition of embodiment 12, wherein the polypeptide comprises an amino acid sequence having at least 85% sequence identity to any one of SEQ ID NOs: 1-16.

14. The pharmaceutical composition of embodiment 12, wherein the polypeptide comprises an amino acid sequence having at least 90% sequence identity to any one of SEQ ID NOs: 1-16.

15. The pharmaceutical composition of embodiment 12, wherein the polypeptide comprises an amino acid sequence having at least 95% sequence identity to any one of SEQ ID NOs: 1-16.

16. The pharmaceutical composition of embodiment 12, wherein the polypeptide comprises an amino acid sequence having 100% sequence identity to any one of SEQ ID NOs: 1-16.

17. The pharmaceutical composition of embodiment 7 or 12, wherein the polypeptide has the sequence of any one of SEQ ID NOs: 33-39.

18. The pharmaceutical composition of any one of embodiments 7-17, further comprising a fluoropyrimidine.

19. A nucleic acid molecule comprising a polynucleotide encoding a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity; and
wherein said nucleic acid molecule further comprises a heterologous promoter operably linked to said polynucleotide.

20. The nucleic acid molecule of embodiment 19, wherein the polypeptide comprises an amino acid sequence having at least 85% sequence identity to any one of SEQ ID NOs: 1-16.

21. The nucleic acid molecule of embodiment 19, wherein the polypeptide comprises an amino acid sequence having at least 90% sequence identity to any one of SEQ ID NOs: 1-16.

22. The nucleic acid molecule of embodiment 19, wherein the polypeptide comprises an amino acid sequence having at least 95% sequence identity to any one of SEQ ID NOs: 1-16.

23. The nucleic acid molecule of embodiment 19, wherein the polypeptide comprises an amino acid sequence having 100% sequence identity to any one of SEQ ID NOs: 1-16.

24. The nucleic acid molecule of embodiment 19, wherein the polypeptide has the sequence of any one of SEQ ID NOs: 33-39.

25. A composition comprising a fluoropyrimidine and a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity.

26. The composition of embodiment 25, wherein the polypeptide comprises an amino acid sequence having at least 85% sequence identity to any one of SEQ ID NOs: 1-16.

27. The composition of embodiment 25, wherein the polypeptide comprises an amino acid sequence having at least 90% sequence identity to any one of SEQ ID NOs: 1-16.

28. The composition of embodiment 25, wherein the polypeptide comprises an amino acid sequence having at least 95% sequence identity to any one of SEQ ID NOs: 1-16.

29. The composition of embodiment 25, wherein the polypeptide comprises an amino acid sequence having 100% sequence identity to any one of SEQ ID NOs: 1-16.

30. A composition comprising a fluoropyrimidine and a nucleic acid molecule encoding a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity. 31. The composition of embodiment 30, wherein the polypeptide comprises an amino acid sequence having at least 85% sequence identity to any one of SEQ ID NOs: 1-16.

32. The composition of embodiment 30, wherein the polypeptide comprises an amino acid sequence having at least 90% sequence identity to any one of SEQ ID NOs: 1-16.

33. The composition of embodiment 30, wherein the polypeptide comprises an amino acid sequence having at least 95% sequence identity to any one of SEQ ID NOs: 1-16.

34. The composition of embodiment 30, wherein the polypeptide comprises an amino acid sequence having 100% sequence identity to any one of SEQ ID NOs: 1-16.

35. The composition of embodiment 25 or 30, wherein the polypeptide has the sequence of any one of SEQ ID NOs: 33-39.

36. A fusion protein comprising: (i) a DNA-binding polypeptide; (ii) a deaminase; and (iii) at least one uracil stabilizing polypeptide (USP) having at least 80% sequence identity to any one of SEQ ID NOs: 1-16.

37. The fusion protein of embodiment 36, wherein at least one USP has at least 85% sequence identity to any one of SEQ ID NOs: 1-16.

38. The fusion protein of embodiment 36, wherein at least one USP has at least 90% sequence identity to any one of SEQ ID NOs: 1-16.

39. The fusion protein of embodiment 36, wherein at least one USP has at least 95% sequence identity to any one of SEQ ID NOs: 1-16.

40. The fusion protein of embodiment 36, wherein at least one USP has 100% sequence identity to any one of SEQ ID NOs: 1-16.

41. The fusion protein of embodiment 36, wherein the USP has the sequence of any one of SEQ ID NOs: 33-39.

42. The fusion protein of embodiment 36 or 41, wherein the deaminase is a cytidine deaminase.

43. The fusion protein of embodiment 42, wherein the cytidine deaminase is an activation-induced cytidine deaminase (AID) or a member of the apolipoprotein B mRNA-editing complex (APOBEC) family of deaminases.

44. The fusion protein of embodiment 43, wherein the cytidine deaminase comprises an amino acid sequence having at least 80% sequence identity to any one of SEQ ID NOs: 47, 48 and 76-94.

45. The fusion protein of embodiment 43, wherein the cytidine deaminase comprises an amino acid sequence having at least 85% sequence identity to any one of SEQ ID NOs: 47, 48 and 76-94.

46. The fusion protein of embodiment 43, wherein the cytidine deaminase comprises an amino acid sequence having at least 90% sequence identity to any one of SEQ ID NOs: 47, 48 and 76-94.

47. The fusion protein of embodiment 43, wherein the cytidine deaminase comprises an amino acid sequence having at least 95% sequence identity to any one of SEQ ID NOs: 47, 48 and 76-94.

48. The fusion protein of embodiment 43, wherein the cytidine deaminase comprises an amino acid sequence having 100% sequence identity to any one of SEQ ID NOs: 47, 48 and 76-94.

49. The fusion protein of any one of embodiments 36-44, wherein the DNA-binding polypeptide is a meganuclease, zinc finger fusion protein, or a TALEN.

50. The fusion protein of any one of embodiments 36-44, wherein the DNA-binding polypeptide is an RNA-guided, DNA-binding polypeptide.

51. The fusion protein of embodiment 50, wherein the RNA-guided, DNA-binding polypeptide is an RNA-guided nuclease polypeptide (RGN).

52. The fusion protein of embodiment 51, wherein the RGN is a Type II CRISPR-Cas polypeptide.

53. The fusion protein of embodiment 51, wherein the RGN is a Type V CRISPR-Cas polypeptide.

54. The fusion protein of embodiment 51, wherein the RGN comprises an amino acid sequence having at least 80% sequence identity to any one of SEQ ID NOs: 40 and 95-142.

55. The fusion protein of embodiment 51, wherein the RGN comprises an amino acid sequence having at least 85% sequence identity to ay one of SEQ ID NOs: 40 and 95-142.

56. The fusion protein of embodiment 51, wherein the RGN comprises an amino acid sequence having at least 90% sequence identity to any one of SEQ ID NOs: 40 and 95-142.

57. The fusion protein of embodiment 51, wherein the RGN comprises an amino acid sequence having at least 95% sequence identity to any one of SEQ ID NOs: 40 and 95-142.

58. The fusion protein of embodiment 51, wherein the RGN comprises an amino acid sequence having 100% sequence identity to any one of SEQ ID NOs: 40 and 95-142.

59. The fusion protein of any one of embodiments 51-58, wherein the RGN is an RGN nickase.

60. The fusion protein of any one of embodiments 51-59, wherein the fusion protein comprises an RGN, a cytidine deaminase, and a USP.

61. The fusion protein of embodiment 60, wherein the fusion protein comprises an RGN having at least 80% sequence identity to any one of SEQ ID NOs: 40, 41, and 95-142, a cytidine deaminase having at least 80% sequence identity to any one of SEQ ID NOs: 47, 48, and 76-94, and a USP having at least 80% sequence identity to any one of SEQ ID NOs: 1-16.

62. The fusion protein of embodiment 60, wherein the fusion protein comprises an RGN having at least 85% sequence identity to any one of SEQ ID NOs: 40, 41, and 95-142, a cytidine deaminase having at least 85% sequence identity to any one of SEQ ID NOs: 47, 48, and 76-94, and a USP having at least 85% sequence identity to any one of SEQ ID NOs: 1-16.

63. The fusion protein of embodiment 60, wherein the fusion protein comprises an RGN having at least 90% sequence identity to any one of SEQ ID NOs: 40, 41, and 95-142, a cytidine deaminase having at least 90% sequence identity to any one of SEQ ID NOs: 47, 48, and 76-94, and a USP having at least 90% sequence identity to any one of SEQ ID NOs: 1-16.

64. The fusion protein of embodiment 60, wherein the fusion protein comprises an RGN having at least 95% sequence identity to any one of SEQ ID NOs: 40, 41, and 95-142, a cytidine deaminase having at least 95% sequence identity to any one of SEQ ID NOs: 47, 48, and 76-94, and a USP having at least 95% sequence identity to any one of SEQ ID NOs: 1-16.

65. The fusion protein of embodiment 60, wherein the fusion protein comprises an RGN having 100% sequence identity to any one of SEQ ID NOs: 40, 41, and 95-142, a cytidine deaminase having 100% sequence identity to any one of SEQ ID NOs: 47, 48, and 76-94, and a USP having 100% sequence identity to any one of SEQ ID NOs: 1-16.

66. The fusion protein of any of embodiments 36-66, wherein the fusion protein further comprises at least one nuclear localization signal (NLS).

67. A nucleic acid molecule comprising a polynucleotide encoding a fusion protein comprising: (i) a DNA-binding polypeptide; (ii) a deaminase; and (iii) at least one uracil stabilizing polypeptide (USP), wherein the USP is encoded by a nucleotide sequence that:
a) has at least 80% sequence identity to any one of SEQ ID NOs: 17-32,
b) is set forth in any one of SEQ ID NOs: 17-32,
c) encodes an amino acid sequence at least 80% identical to SEQ ID NOs: 1-16 and further possesses the sequence of any one of SEQ ID NOs: 33-39,
d) encodes an amino acid sequence at least 80% identical to an amino acid sequence set forth in any one of SEQ ID NOs: 1-16, or
e) encodes an amino acid sequence set forth in any one of SEQ ID NOs: 1-16.

68. The nucleic acid molecule of embodiment 67, wherein the USP is encoded by a nucleotide sequence that:
a) has at least 85% sequence identity to any one of SEQ ID NOs: 17-32,
b) is set forth in any one of SEQ ID NOs: 17-32,
c) encodes an amino acid sequence at least 85% identical to SEQ ID NOs: 1-16 and further possesses the sequence of any one of SEQ ID NOs: 33-39,
d) encodes an amino acid sequence at least 85% identical to an amino acid sequence set forth in any one of SEQ ID NOs: 1-16, or
e) encodes an amino acid sequence set forth in any one of SEQ ID NOs: 1-16.

69. The nucleic acid molecule of embodiment 67, wherein the USP is encoded by a nucleotide sequence that:
a) has at least 90% sequence identity to any one of SEQ ID NOs: 17-32,
b) is set forth in any one of SEQ ID NOs: 17-32,
c) encodes an amino acid sequence at least 90% identical to SEQ ID NOs: 1-16 and further possesses the sequence of any one of SEQ ID NOs: 33-39,
d) encodes an amino acid sequence at least 90% identical to an amino acid sequence set forth in any one of SEQ ID NOs: 1-16, or
e) encodes an amino acid sequence set forth in any one of SEQ ID NOs: 1-16.

70. The nucleic acid molecule of embodiment 67, wherein the USP is encoded by a nucleotide sequence that:
a) has at least 95% sequence identity to any one of SEQ ID NOs: 17-32,
b) is set forth in any one of SEQ ID NOs: 17-32,
c) encodes an amino acid sequence at least 95% identical to SEQ ID NOs: 1-16 and further possesses the sequence of any one of SEQ ID NOs: 33-39,
d) encodes an amino acid sequence at least 95% identical to an amino acid sequence set forth in any one of SEQ ID NOs: 1-16, or
e) encodes an amino acid sequence set forth in any one of SEQ ID NOs: 1-16.

71. The nucleic acid molecule of embodiment 67, wherein the USP is encoded by a nucleotide sequence that:
a) is set forth in any one of SEQ ID NOs: 17-32,
b) encodes an amino acid sequence 100% identical to SEQ ID NOs: 1-16 and further possesses the sequence of any one of SEQ ID NOs: 33-39, or
c) encodes an amino acid sequence set forth in any one of SEQ ID NOs: 1-16.

72. The nucleic acid molecule of embodiment 67, wherein the deaminase is a cytidine deaminase.

73. The nucleic acid molecule of embodiment 72, wherein the cytidine deaminase is an activation-induced cytidine deaminase (AID) or a member of the apolipoprotein B mRNA-editing complex (APOBEC) family of deaminases.

74. The nucleic acid molecule of embodiment 73, wherein the cytidine deaminase comprises an amino acid sequence having at least 80% sequence identity to any one of SEQ ID NOs: 47, 48 and 76-94.

75. The nucleic acid molecule of embodiment 73, wherein the cytidine deaminase comprises an amino acid sequence having at least 85% sequence identity to any one of SEQ ID NOs: 47, 48 and 76-94.

76. The nucleic acid molecule of embodiment 73, wherein the cytidine deaminase comprises an amino acid sequence having at least 90% sequence identity to any one of SEQ ID NOs: 47, 48 and 76-94.

77. The nucleic acid molecule of embodiment 73, wherein the cytidine deaminase comprises an amino acid sequence having at least 95% sequence identity to any one of SEQ ID NOs: 47, 48 and 76-94.

78. The nucleic acid molecule of embodiment 73, wherein the cytidine deaminase comprises an amino acid sequence having 100% sequence identity to any one of SEQ ID NOs: 47, 48 and 76-94.

79. The nucleic acid molecule of any one of embodiments 67-78, wherein the DNA-binding polypeptide is a meganuclease, zinc finger fusion protein, or a TALEN.

80. The nucleic acid molecule of any one of embodiments 67-78, wherein the DNA-binding polypeptide is an RNA-guided, DNA-binding polypeptide.

81. The nucleic acid molecule of embodiment 80, wherein the RNA-guided, DNA-binding polypeptide is an RNA-guided nuclease polypeptide (RGN).

82. The nucleic acid molecule of embodiment 81, wherein the RGN is a Type II CRISPR-Cas polypeptide.

83. The nucleic acid molecule of embodiment 81, wherein the RGN is a Type V CRISPR-Cas polypeptide.

84. The nucleic acid molecule of embodiment 81, wherein the RGN comprises an amino acid sequence having at least 80% sequence identity to any one of SEQ ID NOs: 40 and 95-142.

85. The nucleic acid molecule of embodiment 82, wherein the RGN comprises an amino acid sequence having at least 85% sequence identity to any one of SEQ ID NOs: 40 and 95-142.

86. The nucleic acid molecule of embodiment 81, wherein the RGN comprises an amino acid sequence having at least 90% sequence identity to any one of SEQ ID NOs: 40 and 95-142.

87. The nucleic acid molecule of embodiment 81, wherein the RGN comprises an amino acid sequence having at least 95% sequence identity to any one of SEQ ID NOs: 40 and 95-142.

88. The nucleic acid molecule of embodiment 81, wherein the RGN comprises an amino acid sequence having 100% sequence identity to any one of SEQ ID NOs: 40 and 95-142.

89. The nucleic acid molecule of any one of embodiments 81-88, wherein the RGN is an RGN nickase.

90. The nucleic acid molecule of any one of embodiments 81-89, wherein the fusion protein comprises an RGN, a cytidine deaminase, and a USP.

91. The nucleic acid molecule of embodiment 90, wherein the fusion protein comprises an RGN having at least 80% sequence identity to any one of SEQ ID NOs: 40, 41, and 95-142, a cytidine deaminase having at least 80% sequence identity to any one of SEQ ID NOs: 47, 48, and 76-94, and a USP having at least 80% sequence identity to any one of SEQ ID NOs: 1-16.

92. The nucleic acid molecule of embodiment 90, wherein the fusion protein comprises an RGN having at least 85% sequence identity to any one of SEQ ID NOs: 40, 41, and 95-142, a cytidine deaminase having at least 85% sequence identity to any one of SEQ ID NOs: 47, 48, and 76-94, and a USP having at least 85% sequence identity to any one of SEQ ID NOs: 1-16.

93. The nucleic acid molecule of embodiment 90, wherein the fusion protein comprises an RGN having at least 90% sequence identity to any one of SEQ ID NOs: 40, 41, and 95-142, a cytidine deaminase having at least 90% sequence identity to any one of SEQ ID NOs: 47, 48, and 76-94, and a USP having at least 90% sequence identity to any one of SEQ ID NOs: 1-16.

94. The nucleic acid molecule of embodiment 90, wherein the fusion protein comprises an RGN having at least 95% sequence identity to any one of SEQ ID NOs: 40, 41, and 95-142, a cytidine deaminase having at least 95% sequence identity to any one of SEQ ID NOs: 47, 48, and 76-94, and a USP having at least 95% sequence identity to any one of SEQ ID NOs: 1-16.

95. The nucleic acid molecule of embodiment 90, wherein the fusion protein comprises an RGN having 100% sequence identity to any one of SEQ ID NOs: 40, 41, and 95-142, a cytidine deaminase having 100% sequence identity to any one of SEQ ID NOs: 47, 48, and 76-94, and a USP having 100% sequence identity to any one of SEQ ID NOs: 1-16.

96. The nucleic acid molecule of any of embodiments 67-95, wherein the polynucleotide encoding the fusion protein is operably linked at its 5′ end to a heterologous promoter.

97. The nucleic acid molecule of any of embodiments 67-95, wherein the polynucleotide encoding the fusion protein is operably linked at its 3′ end to a heterologous terminator.

98. The nucleic acid molecule of any of embodiments 67-97, wherein the fusion protein comprises one or more nuclear localization signals.

99. The nucleic acid molecule of any of embodiments 67-98, wherein the fusion protein is codon optimized for expression in a eukaryotic cell.

100. The nucleic acid molecule of any of embodiments 67-99, wherein the fusion protein is codon optimized for expression in a prokaryotic cell.

101. A vector comprising the nucleic acid molecule of any one of embodiments 67-100.

102. A vector comprising the nucleic acid molecule of any one of embodiments 81-95, further comprising at least one nucleotide sequence encoding a guide RNA (gRNA) capable of hybridizing to a target sequence.

103. The vector of embodiment 102, wherein the gRNA is a single guide RNA.

104. The vector of embodiment 102, wherein the gRNA is a dual guide RNA.

105. A cell comprising the fusion protein of any of embodiments 36-66.

106. A cell comprising the fusion protein of any one of embodiments 51-66, wherein the cell further comprises a guide RNA.

107. A cell comprising the nucleic acid molecule of any of embodiments 67-100.

108. A cell comprising the vector of any of embodiments 101 through 104.

109. The cell of any one of embodiments 105-108, wherein the cell is a prokaryotic cell.

110. The cell of any one of embodiments 105-108, wherein the cell is a eukaryotic cell.

111. The cell of embodiment 110, wherein the cell is an insect, avian, or mammalian cell.

112. The cell of embodiment 110, wherein the cell is a plant or fungal cell.

113. A pharmaceutical composition comprising a pharmaceutically acceptable carrier and the nucleic acid molecule of any one of embodiments 19-24 and 67-100, the composition of any one of embodiments 25-35, the fusion protein of any one of embodiments 36-66, the vector of any one of embodiments 101-104, or the cell of any one of embodiments 105-111.

114. A method for making a fusion protein comprising culturing the cell of any one of embodiments 105-112 under conditions in which the fusion protein is expressed.

115. A method for making a fusion protein comprising introducing into a cell the nucleic acid molecule of any of embodiments 67-100 or a vector of any one of embodiments 101-104 and culturing the cell under conditions in which the fusion protein is expressed.

116. The method of embodiment 114 or 115, further comprising purifying said fusion protein.

117. A method for making an RGN fusion ribonucleoprotein complex, comprising introducing into a cell the nucleic acid molecule of any one of embodiments 81-95 and a nucleic acid molecule comprising an expression cassette encoding for a guide RNA, or the vector of any of embodiments 102-104, and culturing the cell under conditions in which the fusion protein and the gRNA are expressed and form an RGN fusion ribonucleoprotein complex.

118. The method of embodiment 117, further comprising purifying said RGN fusion ribonucleoprotein complex.

119. A system for modifying a target DNA molecule comprising a target DNA sequence, said system comprising:
a) a fusion protein comprising an RNA-guided nuclease polypeptide (RGN), a cytidine deaminase, and at least one uracil stabilizing polypeptide (USP), wherein the USP is at least 80% identical to any one of SEQ ID NOs: 1-16, or a nucleotide sequence encoding said fusion protein; and
b) one or more guide RNAs capable of hybridizing to said target DNA sequence or one or more nucleotide sequences encoding the one or more guide RNAs (gRNAs);
wherein said nucleotide sequences encoding the one or more guide RNAs and encoding the fusion protein are each operably linked to a promoter heterologous to said nucleotide sequence;
and
wherein the one or more guide RNAs are capable of forming a complex with the fusion protein in order to direct said fusion protein to bind to said target DNA sequence and modify the target DNA molecule.

120. The system of embodiment 119, wherein the USP is at least 85% identical to any one of SEQ ID NOs: 1-16.

121. The system of embodiment 119, wherein the USP is at least 90% identical to any one of SEQ ID NOs: 1-16.

122. The system of embodiment 119, wherein the USP is at least 95% identical to any one of SEQ ID NOs: 1-16.

123. The system of embodiment 119, wherein the USP is 100% identical to any one of SEQ ID NOs: 1-16.

124. The system of embodiment 119, wherein the USP comprises the sequence set forth in any one of SEQ ID NOs: 33-39.

125. The system of any one of embodiments 119-124, wherein the target DNA sequence is located adjacent to a protospacer adjacent motif (PAM) that is recognized by the RGN.

126. The system of any one of embodiments 119-125, wherein the target DNA molecule is within a cell.

127. The system of embodiment 126, wherein the cell is a eukaryotic cell.

128. The system of embodiment 127, wherein the eukaryotic cell is a plant cell.

129. The system of embodiment 127, wherein the eukaryotic cell is a mammalian cell.

130. The system of embodiment 127, wherein the eukaryotic cell is an insect cell.

131. The system of embodiment 126, wherein the cell is a prokaryotic cell. 132. The system of any one of embodiments 119-131, wherein the RGN of the fusion protein is a Type II CRISPR-Cas polypeptide.

133. The system of any one of embodiments 119-131, wherein the RGN of the fusion protein is a Type V CRISPR-Cas polypeptide.

134. The system of any one of embodiments 119-131, wherein the RGN of the fusion protein is at least 80% identical to any one of SEQ ID NOs: 40 and 95-142.

135. The system of any one of embodiments 119-131, wherein the RGN of the fusion protein is at least 85% identical to any one of SEQ ID NOs: 40 and 95-142.

136. The system of any one of embodiments 119-131, wherein the RGN of the fusion protein is 90% identical to any one of SEQ ID NOs: 40 and 95-142.

137. The system of any one of embodiments 119-131, wherein the RGN of the fusion protein is 95% identical to any one of SEQ ID NOs: 40 and 95-142.

138. The system of any one of embodiments 119-131, wherein the RGN of the fusion protein is 100% identical to any one of SEQ ID NOs: 40 and 95-142.

139. The system of any one of embodiments 119-138, wherein the RGN of the fusion protein is an RGN nickase.

140. The system of any of embodiments 119-139, wherein the cytidine deaminase is at least 80% identical to any one of SEQ ID NOs: 47, 48 and 76-94.

141. The system of any of embodiments 119-139, wherein the cytidine deaminase is at least 85% identical to any one of SEQ ID NOs: 47, 48 and 76-94.

142. The system of any of embodiments 119-139, wherein the cytidine deaminase is at least 90% identical to any one of SEQ ID NOs: 47, 48 and 76-94.

143. The system of any of embodiments 119-139, wherein the cytidine deaminase is at least 95% identical to any one of SEQ ID NOs: 47, 48 and 76-94.

144. The system of any of embodiments 119-139, wherein the cytidine deaminase is 100% identical to any one of SEQ ID NOs: 47, 48 and 76-94.

145. The system of any of embodiments 119-144, wherein the fusion protein comprises a RGN having at least 80% sequence identity to any one of SEQ ID NOs: 40, 41, 95 and 142, a cytidine deaminase having at least 80% sequence identity to any one of EQ ID NOs: 47, 48, and 76-94, and a USP having at least 80% sequence identity to any one of SEQ ID NOs: 1-16.

146. The system of any of embodiments 119-144, wherein the fusion protein comprises a RGN having at least 85% sequence identity to any one of SEQ ID NOs: 40, 41, 95 and 142, a cytidine deaminase having at least 85% sequence identity to any one of EQ ID NOs: 47, 48, and 76-94, and a USP having at least 85% sequence identity to any one of SEQ ID NOs: 1-16.

147. The system of any of embodiments 119-144, wherein the fusion protein comprises a RGN having at least 90% sequence identity to any one of SEQ ID NOs: 40, 41, 95 and 142, a cytidine deaminase having at least 90% sequence identity to any one of EQ ID NOs: 47, 48, and 76-94, and a USP having at least 90% sequence identity to any one of SEQ ID NOs: 1-16.

148. The system of any of embodiments 119-144, wherein the fusion protein comprises a RGN having at least 95% sequence identity to any one of SEQ ID NOs: 40, 41, 95 and 142, a cytidine deaminase having at least 95% sequence identity to any one of EQ ID NOs: 47, 48, and 76-94, and a USP having at least 95% sequence identity to any one of SEQ ID NOs: 1-16.

149. The system of any of embodiments 119-144, wherein the fusion protein comprises a RGN having 100% sequence identity to any one of SEQ ID NOs: 40, 41, 95 and 142, a cytidine deaminase having 100% sequence identity to any one of EQ ID NOs: 47, 48, and 76-94, and a USP having 100% sequence identity to any one of SEQ ID NOs: 1-16.

150. The system of any of embodiments 119-149, wherein the fusion protein comprises one or more nuclear localization signals.

151. The system of any of embodiments 119-150, wherein the fusion protein is codon optimized for expression in a eukaryotic cell.

152. The system of any of embodiments 119-151, wherein nucleotide sequences encoding the one or more guide RNAs and the nucleotide sequence encoding a fusion protein are located on one vector.

153. A method for modifying a target DNA molecule comprising a target DNA sequence, said method comprising delivering a system according to any one of embodiments 119-152 to said target DNA molecule or a cell comprising the target DNA molecule.

154. The method of embodiment 153, wherein said modified target DNA molecule comprises a C>T mutation of at least one nucleotide within the target DNA molecule.

155. The method of embodiment 153, wherein said modified target DNA molecule comprises a C>T mutation of at least one nucleotide within the target DNA sequence.

156. A method for modifying a target DNA molecule comprising a target sequence comprising:
a) assembling an RGN-deaminase-USP ribonucleotide complex in vitro by combining:
   i) one or more guide RNAs capable of hybridizing to the target DNA sequence; and
   ii) a fusion protein comprising an RNA-guided nuclease polypeptide (RGN), a cytidine deaminase, and at least one uracil stabilizing polypeptide (USP), wherein the USP is at least 80% identical to any one of SEQ ID NOs: 1-16;
under conditions suitable for formation of the RGN-deaminase-USP ribonucleotide complex; and
b) contacting said target DNA molecule or a cell comprising said target DNA molecule with the in vitro-assembled RGN-deaminase-USP ribonucleotide complex;
wherein the one or more guide RNAs hybridize to the target DNA sequence, thereby directing said fusion protein to bind to said target DNA sequence and modification of the target DNA molecule occurs.

157. The method of embodiment 156, wherein the USP is at least 85% identical to any one of SEQ ID NOs: 1-16.

158. The method of embodiment 156, wherein the USP is at least 90% identical to any one of SEQ ID NOs: 1-16.

159. The method of embodiment 156, wherein the USP is at least 95% identical to any one of SEQ ID NOs: 1-16.

160. The method of embodiment 156, wherein the USP is 100% identical to any one of SEQ ID NOs: 1-16.

161. The method of embodiment 156, wherein the USP comprises the sequence of any one of SEQ ID NOs: 33-39.

162. The method of any one of embodiments 156-161, wherein said modified target DNA molecule comprises a C>T mutation of at least one nucleotide within the target DNA molecule.

163. The method of any one of embodiments 156-161, wherein said modified target DNA molecule comprises a C>T mutation of at least one nucleotide within the target DNA sequence.

164. The method of any one of embodiments 156-163, wherein the RGN of the fusion protein is a Type II CRISPR-Cas polypeptide.

165. The method of any of embodiments 156-163, wherein the RGN of the fusion protein is a Type V CRISPR-Cas polypeptide.

166. The method of any of embodiments 156-163, wherein the RGN of the fusion protein is at least 80% identical to any one of SEQ ID NOs: 40 and 95-142.

167. The method of any of embodiments 156-163, wherein the RGN of the fusion protein is at least 85% identical to any one of SEQ ID NOs: 40 and 95-142.

168. The method of any of embodiments 156-163, wherein the RGN of the fusion protein is at least 90% identical to any one of SEQ ID NOs: 40 and 95-142.

169. The method of any of embodiments 156-163, wherein the RGN of the fusion protein is at least 95% identical to any one of SEQ ID NOs: 40 and 95-142.

170. The method of any of embodiments 156-163, wherein the RGN of the fusion protein is 100% identical to any one of SEQ ID NOs: 40 and 95-142.

171. The method of any of embodiments 156-170, wherein the RGN of the fusion protein is an RGN nickase.

172. The method of any of embodiments 156-171, wherein the cytidine deaminase is at least 80% identical to any one of SEQ ID NOs: 47, 48 and 76-94.

173. The method of any of embodiments 156-171, wherein the cytidine deaminase is at least 85% identical to any one of SEQ ID NOs: 47, 48 and 76-94.

174. The method of any of embodiments 156-171, wherein the cytidine deaminase is at least 90% identical to any one of SEQ ID NOs: 47, 48 and 76-94.

175. The method of any of embodiments 156-171, wherein the cytidine deaminase is at least 95% identical to any one of SEQ ID NOs: 47, 48 and 76-94.

176. The method of any of embodiments 156-171, wherein the cytidine deaminase is 100% identical to any one of SEQ ID NOs: 47, 48 and 76-94.

177. The method of any one of embodiments 156-176, wherein the fusion protein comprises an RGN having at least 80% sequence identity to any one of SEQ ID NOs: 40, 41, and 95-142, a cytidine deaminase having at least 80% sequence identity to any one of SEQ ID NOs: 47, 48, and 76-94, and a USP having at least 80% sequence identity to any one of SEQ ID NOs: 1-16.

178. The method of any one of embodiments 156-176, wherein the fusion protein comprises an RGN having at least 85% sequence identity to any one of SEQ ID NOs: 40, 41, and 95-142, a cytidine deaminase having at least 85% sequence identity to any one of SEQ ID NOs: 47, 48, and 76-94, and a USP having at least 85% sequence identity to any one of SEQ ID NOs: 1-16.

179. The method of any one of embodiments 156-176, wherein the fusion protein comprises an RGN having at least 90% sequence identity to any one of SEQ ID NOs: 40, 41, and 95-142, a cytidine deaminase having at least 90% sequence identity to any one of SEQ ID NOs: 47, 48, and 76-94, and a USP having at least 90% sequence identity to any one of SEQ ID NOs: 1-16.

180. The method of any one of embodiments 156-176, wherein the fusion protein comprises an RGN having at least 95% sequence identity to any one of SEQ ID NOs: 40, 41, and 95-142, a cytidine deaminase having at least 95% sequence identity to any one of SEQ ID NOs: 47, 48, and 76-94, and a USP having at least 95% sequence identity to any one of SEQ ID NOs: 1-16.

181. The method of any one of embodiments 156-176, wherein the fusion protein comprises an RGN having 100% sequence identity to any one of SEQ ID NOs: 40, 41, and 95-142, a cytidine deaminase having 100% sequence identity to any one of SEQ ID NOs: 47, 48, and 76-94, and a USP having 100% sequence identity to any one of SEQ ID NOs: 1-16.

182. The method of any of embodiments 156-181, wherein the fusion protein comprises one or more nuclear localization signals.

183. The method of any of embodiments 156-182, wherein the fusion protein is codon optimized for expression in a eukaryotic cell.

184. The method of any of embodiments 156-183, wherein said target DNA sequence is located adjacent to a protospacer adjacent motif (PAM).

185. The method of any of embodiments 156-184, wherein the target DNA molecule is within a cell.

186. The method of embodiment 185, wherein the cell is a eukaryotic cell.

187. The method of embodiment 186, wherein the eukaryotic cell is a plant cell.

188. The method of embodiment 186, wherein the eukaryotic cell is a mammalian cell.

189. The method of embodiment 186, wherein the eukaryotic cell is an insect cell.

190. The method of embodiment 185, wherein the cell is a prokaryotic cell.

191. The method of any one of embodiments 185-190, further comprising selecting a cell comprising said modified DNA molecule.

192. A cell comprising a modified target DNA sequence according to the method of embodiment 191.

193. The cell of embodiment 192, wherein the cell is a eukaryotic cell.

194. The cell of embodiment 193, wherein the eukaryotic cell is a plant cell.

195. A plant comprising the cell of embodiment 194.

196. A seed comprising the cell of embodiment 194.

197. The cell of embodiment 193, wherein the eukaryotic cell is a mammalian cell.

198. The cell of embodiment 193, wherein the eukaryotic cell is an insect cell.

199. The cell of embodiment 192, wherein the cell is a prokaryotic cell.

200. A method for producing a genetically modified cell with a correction in a causal mutation for a genetically inherited disease, the method comprising introducing into the cell:
a) a fusion protein comprising an RNA-guided nuclease polypeptide (RGN), a cytidine deaminase, and at least one uracil stabilizing polypeptide (USP), wherein the USP is at least 80% identical to any one of SEQ ID NOs: 1-16, or a polynucleotide encoding said fusion protein, wherein said polynucleotide encoding the fusion protein is operably linked to a promoter to enable expression of the fusion protein in the cell; and
b) one or more guide RNAs (gRNA) capable of hybridizing to a target DNA sequence, or a polynucleotide encoding said gRNA, wherein said polynucleotide encoding the gRNA is operably linked to a promoter to enable expression of the gRNA in the cell;
   whereby the fusion protein and gRNA target to the genomic location of the causal mutation and modify the genomic sequence to remove the causal mutation.

201. The method of embodiment 200, wherein the USP is at least 85% identical to any one of SEQ ID NOs: 1-16.

202. The method of embodiment 200, wherein the USP is at least 90% identical to any one of SEQ ID NOs: 1-16.

203. The method of embodiment 200, wherein the USP is at least 95% identical to any one of SEQ ID NOs: 1-16.

204. The method of embodiment 200, wherein the USP is 100% identical to any one of SEQ ID NOs: 1-16.

205. The method of embodiment 200, wherein said RGN of the fusion protein is a nickase.

206. The method of embodiment 200 or 205, wherein the USP comprises the sequence of any one of SEQ ID NOs: 33-39.

207. The method of any of embodiments 200-206, wherein the genome modification comprises introducing a C>T mutation of at least one nucleotide within the target DNA sequence.

208. The method of any of embodiments 200-207, wherein the cell is an animal cell.

209. The method of embodiment 208, wherein the animal cell is a mammalian cell.

210. The method of embodiment 209, wherein the cell is derived from a dog, cat, mouse, rat, rabbit, horse, sheep, goat, cow, pig, or human.

211. The method of any of embodiments 200-210, wherein the correction of the causal mutation comprises introducing a stop codon.

212. A composition comprising:
a) a fusion protein comprising: (i) a DNA-binding polypeptide; and (ii) a deaminase; or a nucleic acid molecule encoding the fusion protein; and
b) a uracil stabilizing polypeptide (USP) having at least 80% sequence identity to any one of SEQ ID NOs: 1-16; or a nucleic acid molecule encoding the USP.

213. The composition of embodiment 212, wherein the USP has at least 85% sequence identity to any one of SEQ ID NOs: 1-16.

214. The composition of embodiment 212, wherein the USP has at least 90% sequence identity to any one of SEQ ID NOs: 1-16.

215. The composition of embodiment 212, wherein the USP has at least 95% sequence identity to any one of SEQ ID NOs: 1-16.

216. The composition of embodiment 212, wherein the USP has 100% sequence identity to any one of SEQ ID NOs: 1-16.

217. The composition of embodiment 212, wherein the fusion protein further comprises a uracil stabilizing polypeptide (USP) having at least 80% sequence identity to any one of SEQ ID NOs: 1-16.

218. The composition of embodiment 212, wherein the fusion protein further comprises a uracil stabilizing polypeptide (USP) having at least 85% sequence identity to any one of SEQ ID NOs: 1-16.

219. The composition of embodiment 212, wherein the fusion protein further comprises a uracil stabilizing polypeptide (USP) having at least 90% sequence identity to any one of SEQ ID NOs: 1-16.

220. The composition of embodiment 212, wherein the fusion protein further comprises a uracil stabilizing polypeptide (USP) having at least 95% sequence identity to any one of SEQ ID NOs: 1-16.

221. The composition of embodiment 212, wherein the fusion protein further comprises a uracil stabilizing polypeptide (USP) having 100% sequence identity to any one of SEQ ID NOs: 1-16.

222. The composition of embodiment 212, wherein the DNA-binding polypeptide is a meganuclease, zinc finger fusion protein, or a TALEN.

223. The composition of embodiment 212, wherein the DNA-binding polypeptide is an RNA-guided, DNA-binding polypeptide.

224. The composition of embodiment 223, wherein the RNA-guided, DNA-binding polypeptide is an RNA-guided nuclease polypeptide (RGN).

225. The composition of embodiment 224, wherein the RGN is an RGN nickase.

226. A vector comprising a nucleic acid molecule encoding a fusion protein and a nucleic acid molecule encoding a uracil stabilizing polypeptide (USP), wherein said fusion protein comprises a DNA-binding polypeptide and a deaminase, and wherein said USP has at least 80% sequence identity to any one of SEQ ID NOs: 1-16.

227. The vector of embodiment 226, said USP has at least 85% sequence identity to any one of SEQ ID NOs: 1-16.

228. The vector of embodiment 226, said USP has at least 90% sequence identity to any one of SEQ ID NOs: 1-16.

229. The vector of embodiment 226, said USP has at least 95% sequence identity to any one of SEQ ID NOs: 1-16.

230. The vector of embodiment 226, said USP has 100% sequence identity to any one of SEQ ID NOs: 1-16.

231. The vector of embodiment 226, wherein the fusion protein further comprises a uracil stabilizing polypeptide (USP) having at least 80% sequence identity to any one of SEQ ID NOs: 1-16.

232. The vector of embodiment 226, wherein the fusion protein further comprises a uracil stabilizing polypeptide (USP) having at least 85% sequence identity to any one of SEQ ID NOs: 1-16.

233. The vector of embodiment 226, wherein the fusion protein further comprises a uracil stabilizing polypeptide (USP) having at least 90% sequence identity to any one of SEQ ID NOs: 1-16.

234. The vector of embodiment 226, wherein the fusion protein further comprises a uracil stabilizing polypeptide (USP) having at least 95% sequence identity to any one of SEQ ID NOs: 1-16.

235. The vector of embodiment 226, wherein the fusion protein further comprises a uracil stabilizing polypeptide (USP) having 100% sequence identity to any one of SEQ ID NOs: 1-16.

236. The vector of embodiment 226, wherein the DNA-binding polypeptide is a meganuclease, zinc finger fusion protein, or a TALEN.

237. The vector of embodiment 226, wherein the DNA-binding polypeptide is an RNA-guided, DNA-binding polypeptide.

238. The vector of embodiment 237, wherein the RNA-guided, DNA-binding polypeptide is an RNA-guided nuclease polypeptide (RGN).

239. The vector of embodiment 238, wherein the RGN is an RGN nickase.

240. A cell comprising the vector of any one of embodiments 226-239.

241. A cell comprising:
a) a fusion protein comprising: (i) a DNA-binding polypeptide; and (ii) a deaminase; or a nucleic acid molecule encoding the fusion protein; and
b) a uracil stabilizing polypeptide (USP) having at least 80% sequence identity to any one of SEQ ID NOs: 1-16; or a nucleic acid molecule encoding the USP.

242. The cell of embodiment 241, wherein the USP has at least 85% sequence identity to any one of SEQ ID NOs: 1-16.

243. The cell of embodiment 241, wherein the USP has at least 90% sequence identity to any one of SEQ ID NOs: 1-16.

244. The cell of embodiment 241, wherein the USP has at least 95% sequence identity to any one of SEQ ID NOs: 1-16.

245. The cell of embodiment 241, wherein the USP has 100% sequence identity to any one of SEQ ID NOs: 1-16.

246. The cell of embodiment 241, wherein the fusion protein further comprises a uracil stabilizing polypeptide (USP) having at least 80% sequence identity to any one of SEQ ID NOs: 1-16.

247. The cell of embodiment 241, wherein the fusion protein further comprises a uracil stabilizing polypeptide (USP) having at least 85% sequence identity to any one of SEQ ID NOs: 1-16.

248. The cell of embodiment 241, wherein the fusion protein further comprises a uracil stabilizing polypeptide (USP) having at least 90% sequence identity to any one of SEQ ID NOs: 1-16.

249. The cell of embodiment 241, wherein the fusion protein further comprises a uracil stabilizing polypeptide (USP) having at least 95% sequence identity to any one of SEQ ID NOs: 1-16.

250. The cell of embodiment 241, wherein the fusion protein further comprises a uracil stabilizing polypeptide (USP) having 100% sequence identity to any one of SEQ ID NOs: 1-16.

251. The cell of embodiment 241, wherein the DNA-binding polypeptide is a meganuclease, zinc finger fusion protein, or a TALEN.

252. The cell of embodiment 241, wherein the DNA-binding polypeptide is an RNA-guided, DNA-binding polypeptide.

253. The cell of embodiment 252, wherein the RNA-guided, DNA-binding polypeptide is an RNA-guided nuclease polypeptide (RGN).

254. The cell of embodiment 253, wherein the RGN is an RGN nickase.

The following examples are offered by way of illustration and not by way of limitation.

### EXPERIMENTAL

### Example 1: Uracil Stabilizing Polypeptides

Amino acid sequences for the Uracil Stabilizing Polypeptides (USPs) of the invention are provided as SEQ ID NOs: 1-16, as shown in Table 1. All USPs disclosed are from Staphylococcus spp and range from 112 to 116 amino acids in length. The polypeptides of the USPs described herein possess 16.4% to 20.7% negative charges, with an expected pI of 3.86-4.55, except for APG05963 which has an expected pI of 5.26.

USPs APG06351, APG03399, APG04638, APG09242, APG02463, APG04080, APG01791, APG04001, and APG03327 share a unique consensus C-terminus sequence of “KEGGNDHE” (SEQ ID NO: 33). USPs APG05198 and APG05756 share a unique C-terminus sequence of “EKENYNNE” (SEQ ID NO: 34). APG05963 possesses a unique C-terminus sequence of “EKEKHKNE” (SEQ ID NO: 35); APG06702 possesses a unique C-terminus sequence of “DKGDDNHD” (SEQ ID NO: 36); APG05316 possesses a unique C-terminus sequence of “QKGGQ” (SEQ ID NO: 37); APG09230 possesses a unique C-terminus sequence of “KGENKYE” (SEQ ID NO: 38); and APG04100 possesses a unique C-terminus sequence of “KQGENNHE” (SEQ ID NO: 39).

TABLE 1
Uracil stabilizing polypeptides

| | SEQ ID |
|---|---|
| USP ID | NO |
| APG03399 | 1 |
| APG06702 | 2 |
| APG05198 | 3 |
| APG04638 | 4 |
| APG06351 | 5 |
| APG05963 | 6 |
| APG09230 | 7 |
| APG04100 | 8 |
| APG04001 | 9 |
| APG02463 | 10 |
| APG09242 | 11 |
| APG04080 | 12 |
| APG05316 | 13 |
| APG03327 | 14 |
| APG01791 | 15 |
| APG05756 | 16 |

### Example 2: USP Fusion Proteins Exhibit Increased Base Editing Activity in Mammalian Cells

Residues predicted to deactivate the RuvC domain of the RGN APG07433.1 (SEQ ID NO: 40; PCT publication WO 2019/236566, incorporated by reference herein) were identified and the RGN was modified to a nickase variant (nAPG07433.1; SEQ ID NO: 41). Fusion proteins comprising a cytidine deaminase, namely APG09980 (SEQ ID NO: 47; see PCT/US2019/068079, incorporated by reference herein) or APG07386CTD (SEQ ID NO: 48; see PCT/US2019/068079), were produced. Of the USPs in Table 1, three were selected for assaying for activity in fusion proteins, namely APG03399, APG06702, and APG05198. Deaminase, USP, and nRGN nucleotide sequences codon optimized for expression were synthesized as fusion proteins with an N-terminal nuclear localization tag and cloned into the pTwist CMV (Twist Biosciences) expression plasmid. A fusion protein lacking a USP of the invention comprises, starting at the amino terminus, the SV40 NLS (SEQ ID NO: 42) operably linked at the C-terminal end to 3X FLAG Tag (SEQ ID NO: 43), operably linked at the C-terminal end to a deaminase, operably linked at the C-terminal end to a peptide linker (SEQ ID NO: 44), operably linked at the C-terminal end to the nRGN (for example, nAPG07433.1, which is SEQ ID NO: 41), finally operably linked at the C-terminal end to the nucleoplasmin NLS (SEQ ID NO: 45). A fusion protein comprising a USP of the invention comprises, starting at the amino terminus, the SV40 NLS (SEQ ID NO: 42) operably linked at the C-terminal end to 3X FLAG Tag (SEQ ID NO: 43), operably linked at the C-terminal end to a deaminase, operably linked at the C-terminal end to a peptide linker (SEQ ID NO: 44), operably linked at the C-terminal end to the nRGN (for example, SEQ ID NO: 41), operably linked at the C-terminal end to a second linker sequence (SEQ ID NO: 46), operably linked at the C-terminal end to a USP of the invention, finally operably linked at the C-terminal end to the nucleoplasmin NLS (SEQ ID NO: 45). Table 2 shows the fusion proteins produced and tested for activity. All fusion proteins comprise at least one NLS and a 3X FLAG Tag, as described above.

TABLE 2
Fusion proteins assayed for C > T Editing

| | SEQ ID |
|---|---|
| Fusion protein | NO |
| APG09980-nAPG07433.1 | 49 |
| APG09980-nAPG07433.1-APG03399 | 50 |
| APG09980-nAPG07433.1-APG06702 | 51 |
| APG09980-nAPG07433.1-APG05198 | 52 |
| APG07386-CTD-nAPG07433.1 | 53 |
| APG07386-CTD-nAPG07433.1-APG03399 | 54 |
| APG07386-CTD-nAPG07433.1-APG06702 | 55 |
| APG07386-CTD-nAPG07433.1-APG05198 | 56 |

Expression plasmids comprising an expression cassette encoding for a sgRNA were also produced. Human genomic target sequences and the sgRNA sequences for guiding the fusion proteins to the genomic targets are indicated in Table 3. The genomic loci for each target sequence is also indicated.

TABLE 3
guide RNA sequences

| | Target | sgRNA | Genomic |
|---|---|---|---|
| sgRNA ID | sequence | sequence | locus |
| SGN000143 | 57 | 63 | 69 |
| SGN000169 | 58 | 64 | 70 |
| SGN000173 | 59 | 65 | 71 |
| SGN000929 | 60 | 66 | 72 |
| SGN000930 | 61 | 67 | 73 |
| SGN001101 | 62 | 68 | 74 |

500 ng of plasmid comprising an expression cassette comprising a coding sequence for a fusion protein shown in Table 2 and 500 ng of plasmid comprising an expression cassette encoding for an sgRNA shown in Table 3 were co-transfected into HEK293FT cells at 75-90% confluency in 24-well plates using

Lipofectamine 2000 reagent (Life Technologies). Cells were then incubated at 37° C. for 72 h. Following incubation, genomic DNA was then extracted using NucleoSpin 96 Tissue (Macherey-Nagel) following the manufacturer's protocol. The genomic region flanking the targeted genomic site was PCR amplified and products were purified using ZR-96 DNA Clean and Concentrator (Zymo Research) following the manufacturer's protocol. The purified PCR products were then sent for Next Generation Sequencing on

Illumina MiSeq (2×250). Results were analyzed for indel formation or specific cytosine mutation out to +30 nucleotides, where the last nucleotide at the 3′ end of the target sequence described in Table 3 is +1, and wherein the +30 nucleotide is 29 nucleotides upstream or 5′ from the +1 nucleotide in the target sequence set forth in SEQ ID NOs: 57-62 (the target sequences of SEQ ID NOs: 57-62 are indicated as lower-case text within the genomic locus sequences set forth in SEQ ID NOs: 69-74, respectively).

Tables 4 through 15 show cytidine base editing for each combination of a fusion protein from Table 2 and a guide RNA from Table 3. Fusion proteins are identified by their SEQ ID NO. The numbering of cytidines (Cs) in Tables 4-15 are as in the preceding paragraph wherein the last nucleotide at the 3′ end of the target sequence is +1 and the numbering proceeds in the 3′ to 5′ direction in the target sequence and corresponding genomic locus sequence. Interestingly, when comparing the activity of a deaminase-nRGN fusion protein with that of a corresponding deaminase-nRGN plus a USP, using the same guide RNA, the conversion of a cytidine to the desired thymidine is higher, with less conversion to an adenosine or guanosine.

TABLE 4
C > N Editing Rate using deaminase
APG09980 and guide SGN000143

| Fusion Protein | | | | | | | |
|---|---|---|---|---|---|---|---|
| (SEQ ID NO) | | C10 | C15 | C20 | C21 | C22 | C25 |
| 49 | A | 2.8 | 0.1 | 0 | 0.2 | 0.6 | 0.1 |
| | G | 1.1 | 0.1 | 0.1 | 0.1 | 0.2 | 0 |
| | T | 11 | 0.4 | 0.2 | 0.3 | 2 | 0.7 |
| 50 | A | 0 | 0 | 0 | 0 | 0 | 0 |
| | G | 0 | 0 | 0 | 0 | 0 | 0 |
| | T | 23.5 | 1.4 | 1.7 | 2 | 4.6 | 0.7 |
| 51 | A | 0.1 | 0 | 0 | 0 | 0 | 0 |
| | G | 0 | 0 | 0 | 0 | 0 | 0 |
| | T | 16.3 | 1.2 | 1.5 | 1.6 | 3.1 | 0.6 |
| 52 | A | 0.2 | 0 | 0 | 0 | 0 | 0 |
| | G | 0 | 0 | 0 | 0 | 0 | 0 |
| | T | 21.4 | 1.6 | 1.1 | 1.4 | 2.5 | 0.7 |

The results of Table 4 show that the rate of C>T editing at position C10 increased in samples with a USP.

TABLE 5
C > N Editing Rate using deaminase
APG07386 and guide SGN000143

| Fusion Protein | | | | | | | |
|---|---|---|---|---|---|---|---|
| (SEQ ID NO.) | | C10 | C15 | C20 | C21 | C22 | C25 |
| 53 | A | 0.1 | 0.4 | 0 | 0.5 | 1 | 0 |
| | G | 0 | 0.8 | 0 | 0 | 0.1 | 0 |
| | T | 0.5 | 4 | 0.3 | 0.7 | 3.8 | 0 |
| 54 | A | 0 | 0 | 0 | 0 | 0.1 | 0 |
| | G | 0 | 0 | 0 | 0 | 0 | 0 |
| | T | 1 | 4.5 | 1 | 1.7 | 3.1 | 0.1 |
| 55 | A | 0 | 0.1 | 0 | 0 | 0.1 | 0 |
| | G | 0 | 0 | 0 | 0 | 0 | 0 |
| | T | 1.6 | 9.7 | 2.5 | 3.2 | 5.8 | 0.2 |
| 56 | A | 0 | 0.1 | 0 | 0 | 0 | 0 |
| | G | 0 | 0 | 0 | 0 | 0 | 0 |
| | T | 1.9 | 10.2 | 2.6 | 3.1 | 7 | 0.2 |

The results of Table 5 show that the rate of C>T editing at position C15 increased in samples with a USP.

TABLE 6
C > N Editing Rate using deaminase
APG09980 and guide SGN000169

| Fusion Protein | | | | | | | |
|---|---|---|---|---|---|---|---|
| (SEQ ID NO.) | | C9 | C13 | C15 | C18 | C20 | C23 |
| 49 | A | 0.5 | 0.8 | 3.6 | 0.3 | 1.3 | 1.3 |
| | G | 1.5 | 1.8 | 8.3 | 0 | 0.2 | 0.1 |
| | T | 1.5 | 23.9 | 16.3 | 0.3 | 8.6 | 4.9 |
| 50 | A | 0 | 0 | 0.6 | 0 | 0.1 | 0.2 |
| | G | 0.2 | 0 | 2.5 | 0 | 0 | 0 |
| | T | 7 | 38.1 | 35.8 | 0.5 | 16.6 | 13.4 |
| 51 | A | 0.2 | 0.3 | 1.3 | 0.2 | 0.3 | 0.4 |
| | G | 0.6 | 0.6 | 2.1 | 0.1 | 0.1 | 0 |
| | T | 9.2 | 42.4 | 40.8 | 0.8 | 20.4 | 16.6 |
| 52 | A | 0.8 | 1 | 2.4 | 0.7 | 0.6 | 1.5 |
| | G | 0.5 | 1.7 | 3.7 | 0.5 | 0.5 | 0.2 |
| | T | 8.5 | 37.8 | 35.7 | 1.2 | 17.1 | 14.9 |

The results of Table 6 show that the rate of C>T formation at positions C13 and C15 increased with the addition of a USP.

TABLE 7
C > N Editing Rate using deaminase
APG07386 and guide SGN000169

| Fusion Protein | | | | | | | |
|---|---|---|---|---|---|---|---|
| (SEQ ID NO.) | | C9 | C13 | C15 | C18 | C20 | C23 |
| 53 | A | 0.8 | 0.7 | 2 | 0 | 0.5 | 0.3 |
| | G | 5.4 | 0.8 | 10.2 | 0.1 | 0.1 | 0 |
| | T | 3.5 | 5.1 | 11.2 | 0.3 | 2.6 | 2.4 |
| 54 | A | 0.1 | 0.5 | 0.8 | 0 | 0.3 | 0.2 |
| | G | 0.7 | 0.2 | 2.5 | 0.2 | 0.1 | 0 |
| | T | 8.5 | 15.9 | 24.8 | 1.5 | 6.4 | 7.1 |
| 55 | A | 0.3 | 0.5 | 1.3 | 0.4 | 0.2 | 0.4 |
| | G | 2 | 0.5 | 3.5 | 0.1 | 0.2 | 0.1 |
| | T | 14.3 | 22.7 | 37.1 | 2 | 9.2 | 7.9 |
| 56 | A | 0.1 | 0.1 | 0.7 | 0 | 0.2 | 0 |
| | G | 0.7 | 0 | 2.9 | 0 | 0 | 0 |
| | T | 14.7 | 24 | 36.4 | 1.3 | 8.4 | 8.9 |

The results of Table 7 show that the rate of C>T formation at positions C13 and C15 increased with the addition of a USP.

TABLE 8
C > N Editing Rate using deaminase
APG09980 and guide SGN000930

| Fusion Protein | | | | |
|---|---|---|---|---|
| (SEQ ID NO.) | | C17 | C19 | C22 |
| 49 | A | 0.7 | 0.4 | 1.4 |
| | G | 34.5 | 0.4 | 0 |
| | T | 2.6 | 2.4 | 2.2 |
| 50 | A | 0.3 | 0.1 | 0.3 |
| | G | 8.6 | 0.3 | 0 |
| | T | 33 | 2.8 | 3.2 |
| 51 | A | 0.2 | 0.1 | 0.2 |
| | G | 5.1 | 0.1 | 0.1 |
| | T | 35.7 | 3.6 | 3.3 |
| 52 | A | 0.4 | 0.3 | 0.9 |
| | G | 11.2 | 0 | 0 |
| | T | 23.7 | 2.5 | 2.5 |

The results of Table 8 show that the rate of C>G editing at position C17 decreased in all samples with a USP in favor of C>T changes.

TABLE 9
C > N Editing Rate using deaminase
APG07386 and guide SGN000930

| Fusion Protein | | | | |
|---|---|---|---|---|
| (SEQ ID NO.) | | C17 | C19 | C22 |
| 53 | A | 0.2 | 0 | 0.4 |
| | G | 14.7 | 0 | 0.4 |
| | T | 1.1 | 0.2 | 2.2 |
| 54 | A | 0 | 0 | 0 |
| | G | 1.4 | 0 | 0 |
| | T | 9.1 | 0.4 | 3.7 |
| 55 | A | 0.4 | 0.1 | 0.3 |
| | G | 2.6 | 0.2 | 0.2 |
| | T | 11.8 | 0.9 | 4.5 |
| 56 | A | 0.1 | 0.2 | 0 |
| | G | 2.7 | 0 | 0.1 |
| | T | 17.4 | 0.8 | 6.7 |

The results of Table 9 show that the C>G editing at position C17 decreased in all samples with a USP in favor of C>T changes.

TABLE 10
C > N Editing Rate using deaminase APG09980 and guide SGN00173

| Fusion Protein | | | | | | | | | | | |
|---|---|---|---|---|---|---|---|---|---|---|---|
| (SEQ ID NO.) | | C1 | C2 | C3 | C4 | C7 | C8 | C10 | C11 | C17 | C20 |
| 49 | A | 0 | 0 | 0.1 | 0.3 | 3.1 | 6.9 | 0.3 | 0 | 3.4 | 0.1 |
| | G | 0 | 0.1 | 0 | 0.2 | 0.3 | 1 | 0.1 | 0.4 | 3.4 | 0.1 |
| | T | 0.1 | 0 | 0 | 2.5 | 11.1 | 12.3 | 1.7 | 0.2 | 12.1 | 1.7 |
| 50 | A | 0 | 0.1 | 0 | 0.1 | 0.9 | 2.9 | 0 | 0.4 | 1.5 | 0 |
| | G | 0.1 | 0.1 | 0 | 0 | 0.5 | 0.8 | 0.4 | 0.3 | 1.9 | 0.2 |
| | T | 0.1 | 0.1 | 0.3 | 14 | 39 | 39.3 | 28.7 | 13 | 31.1 | 12 |
| 51 | A | 0 | 0 | 0 | 0 | 1.2 | 1.6 | 0 | 0 | 0.9 | 0 |
| | G | 0 | 0 | 0 | 0 | 0 | 1.4 | 0.1 | 0 | 2.2 | 0.1 |
| | T | 0 | 0 | 0.3 | 10.4 | 33.5 | 33.4 | 23.8 | 10.9 | 26.8 | 10.3 |
| 52 | A | 0 | 0 | 0 | 0 | 1.8 | 1.2 | 0.3 | 0 | 1.5 | 0 |
| | G | 0 | 0 | 0 | 0 | 0 | 0.8 | 0 | 0.5 | 2.7 | 0 |
| | T | 0 | 0 | 0.6 | 10.5 | 33.1 | 36.8 | 26.7 | 13.2 | 28.6 | 9.6 |

The results of Table 10 show that the rate of C>T formation at positions C4, C7, C8, C10, C11, C17 and C20 increased with the addition of a USP.

TABLE 11
C > N Editing Rate using deaminase APG07386 and guide SGN00173

| Fusion Protein | | | | | | | | | | | |
|---|---|---|---|---|---|---|---|---|---|---|---|
| (SEQ ID NO.) | | C1 | C2 | C3 | C4 | C7 | C8 | C10 | C11 | C17 | C20 |
| 53 | A | 0 | 0 | 0 | 0.1 | 0.4 | 1.1 | 0 | 2.5 | 2.8 | 0 |
| | G | 0 | 0 | 0 | 0 | 0.1 | 0.3 | 0 | 0.5 | 2 | 0 |
| | T | 0 | 0 | 0 | 0.5 | 1.4 | 2.5 | 0 | 7 | 11.3 | 0.7 |
| 54 | A | 0 | 0 | 0 | 0 | 0 | 0.1 | 0 | 0.7 | 0.8 | 0 |
| | G | 0 | 0 | 0 | 0 | 0 | 0 | 0 | 0 | 0.6 | 0 |
| | T | 0 | 0 | 0 | 1.6 | 6.1 | 8.6 | 2.2 | 13.3 | 23 | 1.8 |
| 55 | A | 0 | 0 | 0 | 0 | 0.2 | 0 | 0 | 1 | 0.9 | 0 |
| | G | 0 | 0 | 0 | 0 | 0 | 0.3 | 0.1 | 0.1 | 0.5 | 0 |
| | T | 0 | 0 | 0.2 | 2.2 | 9.1 | 11.2 | 3.2 | 13 | 21.3 | 2 |
| 56 | A | 0 | 0 | 0 | 0 | 0 | 0 | 0 | 0.9 | 0.8 | 0 |
| | G | 0 | 0 | 0 | 0 | 0 | 0.1 | 0 | 0.2 | 0.5 | 0 |
| | T | 0 | 0 | 0.1 | 2.7 | 12.3 | 15 | 4.3 | 18.3 | 27.1 | 1.9 |

The results of Table 11 show that the rate of C>T formation at positions C7, C8, C11 and C17 increased with the addition of a USP.

TABLE 12
C > N Editing Rate using deaminase
APG09980 and guide SGN000929

| Fusion Protein | | | | | |
|---|---|---|---|---|---|
| (SEQ ID NO.) | | C6 | C9 | C13 | C23 |
| 49 | A | 0.2 | 1.1 | 0.2 | 5.2 |
| | G | 0.2 | 1.5 | 1.4 | 20.1 |
| | T | 1.2 | 5.2 | 1 | 7.4 |
| 50 | A | 0 | 0.1 | 0 | 1.3 |
| | G | 0 | 0.1 | 0 | 3.3 |
| | T | 2.2 | 9.4 | 3.2 | 30.8 |
| 51 | A | 0 | 0.2 | 0 | 0.8 |
| | G | 0 | 0.1 | 0 | 1.6 |
| | T | 1.5 | 9.2 | 3.5 | 34 |
| 52 | A | 0.5 | 0.6 | 0 | 1.8 |
| | G | 0.1 | 0.5 | 0 | 6.5 |
| | T | 1.6 | 8.6 | 2.7 | 22.3 |

The results of Table 12 show that the rate of C>T formation at position C23 increased with the addition of a USP.

TABLE 13
C > N Editing Rate using deaminase
APG07386 and guide SGN000929

| Fusion Protein | | | | | |
|---|---|---|---|---|---|
| (SEQ ID NO.) | | C6 | C9 | C13 | C23 |
| 53 | A | 1.5 | 0 | 0 | 1.5 |
| | G | 0.6 | 0 | 0.1 | 5.2 |
| | T | 5 | 0.3 | 0.1 | 2.1 |
| 54 | A | 0 | 0 | 0 | 0.1 |
| | G | 0 | 0 | 0 | 0.1 |
| | T | 4.2 | 0.3 | 0 | 4.6 |
| 55 | A | 0.2 | 0 | 0.1 | 0 |
| | G | 0 | 0 | 0 | 1.4 |
| | T | 10.4 | 0.9 | 0.3 | 8.2 |
| 56 | A | 0.1 | 0 | 0 | 0.2 |
| | G | 0.2 | 0 | 0 | 0.5 |
| | T | 10.8 | 0.6 | 0.4 | 8.5 |

The results of Table 13 show that the rate of C>T formation at positions C6 and C23 increased with the addition of a USP.

TABLE 14
C > N Editing Rate using deaminase
APG09980 and guide SGN001101

| Fusion Protein | | | | | |
|---|---|---|---|---|---|
| (SEQ ID NO.) | | C2 | C6 | C7 | C18 |
| 49 | A | 0.1 | 1.8 | 0.4 | 3.3 |
| | G | 0 | 2.8 | 0.1 | 18.5 |
| | T | 0.1 | 6.5 | 1.8 | 5.3 |
| 50 | A | 0.2 | 0.3 | 0.2 | 0.6 |
| | G | 0.1 | 0.1 | 0.2 | 2.1 |
| | T | 0.1 | 9.1 | 0.7 | 25.4 |
| 51 | A | 0.2 | 0.1 | 0.1 | 0.3 |
| | G | 0.1 | 0.1 | 0 | 2.4 |
| | T | 0.1 | 7.9 | 0.7 | 21.8 |
| 52 | A | 0.2 | 0.9 | 0.2 | 1.2 |
| | G | 0 | 0.8 | 0 | 4.8 |
| | T | 0.2 | 7.7 | 1.3 | 19.4 |

The results of Table 14 show that the rate of C>T formation at position C18 increased with the addition of a USP.

TABLE 15
C > N Editing Rate using deaminase
APG07386 and guide SGN001101

| Fusion Protein | | | | | |
|---|---|---|---|---|---|
| (SEQ ID NO.) | | C2 | C6 | C7 | C18 |
| 53 | A | 0.1 | 0 | 4 | 2.2 |
| | G | 0 | 0 | 2.1 | 11 |
| | T | 0 | 0 | 10.4 | 3.9 |
| 54 | A | 0.1 | 0 | 0.1 | 0 |
| | G | 0 | 0 | 0.1 | 0.6 |
| | T | 0 | 1.7 | 8.5 | 6.7 |
| 55 | A | 0.2 | 0.1 | 0.6 | 0.3 |
| | G | 0 | 0 | 0.5 | 1.4 |
| | T | 0 | 1.7 | 10.7 | 9.2 |
| 56 | A | 0.1 | 0 | 0.3 | 0.2 |
| | G | 0 | 0 | 0.4 | 1.4 |
| | T | 0 | 1.9 | 12.1 | 9.8 |

The results of Table 15 show that the rate of C>T formation at position C18 increased with the addition of a USP. The rate of C>G conversion was decreased at position C18 with the addition of a USP.

Tables 16 and 17 show the rate of indel formation for each fusion protein/guide combination tested. The fusion protein is indicated by SEQ ID NO. The data indicates that the fusion proteins comprising a USP described herein decreased the rate of indel formation at all target genomic locations tested.

TABLE 16
Insertion and Deletion Rate with APG09980 and USPs

| | SEQ ID | SEQ ID | SEQ ID | SEQ ID |
|---|---|---|---|---|
| sgRNA ID | NO: 49 | NO: 50 | NO: 51 | NO: 52 |
| SGN000143 | 2.78 | 0 | 0 | 0.62 |
| SGN000169 | 18.02 | 1.62 | 3.89 | 9.1 |
| SGN000173 | 27.46 | 7.86 | 7.17 | 7.48 |
| SGN000929 | 3.49 | 0.2 | 0.65 | 2.36 |
| SGN000930 | 6.03 | 0.81 | 0.65 | 1.65 |
| SGN001101 | 2.5 | 1.66 | 0.92 | 1.86 |

TABLE 17
Insertion and Deletion Rate with APG07386-CTD and USPs

| | SEQ ID | SEQ ID | SEQ ID | SEQ ID |
|---|---|---|---|---|
| sgRNA ID | NO: 53 | NO: 54 | NO: 55 | NO: 56 |
| SGN000143 | 0.13 | 0.05 | 0 | 0.06 |
| SGN000169 | 5.6 | 1.38 | 3.11 | 0.32 |
| SGN000173 | 13.8 | 0.99 | 3.49 | 2.57 |
| SGN000929 | 0.5 | 0 | 0.3 | 0.16 |
| SGN000930 | 1.7 | 0 | 0.23 | 0.31 |
| SGN001101 | 1.78 | 0 | 0.48 | 0.39 |

### Example 3: Testing Different Delivery Formats

To determine if the base editors are capable of delivery in different formats, mRNA delivery was tested with primary T-cells. Purified CD3+ T-cells or PBMCs were thawed, activated using CD3/CD28 beads (ThermoFisher) for 3 days, then nucleofected using the Lonza 4D-Nucleofector X unit and Nucleocuvette strips. The P3 Primary Cell kit was used for both mRNA and RNP delivery. Cells were transfected using the EO-115 and EH-115 programs for mRNA and RNP delivery respectively. Cells were cultured in CTS OpTimizer T cell expansion medium (ThermoFisher) containing IL-2, IL-7, and IL-15 (Miltenyi Biotec) for 4 days post nucleofection before being harvested using a Nucleospin Tissue genomic DNA isolation kit (Machery Nagel).

Amplicons surrounding the editing sites were generated by PCR and subjected to NGS sequencing using the Illumina Nexterra platform using 2×250 bp paired end sequencing. The estimated base editing rate was determined by calculating the overall substitution rate for each sample. The average and number of samples for each guide tested are shown in Tables 18 and 19 below.

APG09980-nAPG07433.1-APG03399 and APG05840-nAPG07433.1-APG03399 when delivered by mRNA show high rates of base editing as several targets. There are very low rates of indel formation despite the high substitution rate, due to the incorporation of USP2 in the base editing construct.

TABLE 18
Average base editing rate for APG09980-nAPG07433.1-APG03399

| | Gene | | Average % | Average % | |
|---|---|---|---|---|---|
| Fusion Construct | Name | SGN | Substitutions | Indels | N |
| APG09980- | Gene 1 | SGN000754 | 23.32917436 | 1.128931 | 6 |
| nAPG07433.1-APG03399 | | | | | |
| APG09980- | Gene 1 | SGN000755 | 59.37254849 | 7.1037823 | 4 |
| nAPG07433.1-APG03399 | | | | | |
| APG09980- | Gene 2 | SGN001061 | 13.60100568 | 0.4214674 | 3 |
| nAPG07433.1-APG03399 | | | | | |
| APG09980- | Gene 2 | SGN001062 | 26.9304354 | 4.3225871 | 4 |
| nAPG07433.1-APG03399 | | | | | |
| APG09980- | Gene 2 | SGN001063 | 75.27761104 | 0.8163273 | 4 |
| nAPG07433.1-APG03399 | | | | | |
| APG09980- | Gene 2 | SGN001064 | 72.94658862 | 1.0468487 | 3 |
| nAPG07433.1-APG03399 | | | | | |

TABLE 19
Average base editing rate for APG05840-nAPG07433.1-APG03399

| | Gene | | Average % | Average % | |
|---|---|---|---|---|---|
| Fusion Construct | Name | SGN | Substitutions | Indels | N |
| APG05840- | Gene 1 | SGN000754 | 57.7775198 | 5.14624384 | 4 |
| nAPG07433.1-APG03399 | | | | | |
| APG05840- | Gene 1 | SGN000755 | 68.352455 | 4.98538891 | 3 |
| nAPG07433.1-APG03399 | | | | | |
| APG05840- | Gene 2 | SGN001061 | 14.6830209 | 0 | 2 |
| nAPG07433.1-APG03399 | | | | | |
| APG05840- | Gene 2 | SGN001062 | 39.7312597 | 2.9887885 | 4 |
| nAPG07433.1-APG03399 | | | | | |
| APG05840- | Gene 2 | SGN001063 | 70.4564399 | 0.25727852 | 4 |
| nAPG07433.1-APG03399 | | | | | |
| APG05840- | Gene 2 | SGN001064 | 53.2112842 | 1.98008536 | 3 |
| nAPG07433.1-APG03399 | | | | | |

1. A fusion polypeptide comprising (A) a first amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said first amino acid sequence encodes a polypeptide that has uracil stabilizing activity and (B) a second amino acid sequence that is heterologous to the first amino acid sequence.

2. The fusion polypeptide of claim 1, wherein the first amino acid sequence comprises the sequence of any one of SEQ ID NOs: 33-39.

3.-6. (canceled)

7. A nucleic acid molecule comprising a polynucleotide sequence encoding a polypeptide comprising an amino acid sequence having:
a) at least 80% sequence identity to any one of SEQ ID NOs: 1, 2, 4, 5, and 7-15;
b) at least 81% sequence identity to SEQ ID NO: 3 or 16; or
c) at least 82% sequence identity to SEQ ID NO: 6;
wherein said polypeptide has uracil stabilizing activity; and
wherein said nucleic acid molecule further comprises a heterologous promoter operably linked to said polynucleotide sequence.

8.-11. (canceled)

12. A fusion protein comprising: (i) a DNA-binding polypeptide; (ii) a deaminase;
and (iii) at least one uracil stabilizing polypeptide (USP) having at least 80% sequence identity to any one of SEQ ID NOs: 1-16.

13. The fusion protein of claim 12, wherein the USP has the sequence of any one of SEQ ID NOs: 33-39.

14.-15. (canceled)

16. The fusion protein of claim 12, wherein the deaminase comprises an amino acid sequence having at least 80% sequence identity to any one of SEQ ID NOs: 47, 48 and 76-94.

17.-18. (canceled)

19. The fusion protein of claim 12, wherein the DNA-binding polypeptide is an RNA-guided nuclease polypeptide (RGN).

20.-21. (canceled)

22. The fusion protein of claim 19, wherein the RGN comprises an amino acid sequence having at least 80% sequence identity to any one of SEQ ID NOs: 40 and 95-142.

23. The fusion protein of claim 19, wherein the RGN is an RGN nickase.

24.-25. (canceled)

26. The fusion protein of claim 12, wherein the fusion protein further comprises at least one nuclear localization signal (NLS).

27.-48. (canceled)

49. A cell comprising the fusion protein of claim 12.

50. A cell comprising the fusion protein of claim 19, wherein the cell further comprises a guide RNA.

51.-62. (canceled)

63. A system for modifying a target DNA molecule comprising a target DNA sequence, said system comprising:
a) the fusion protein of claim 19, or a nucleotide sequence encoding said fusion protein; and
b) one or more guide RNAs capable of hybridizing to said target DNA sequence or one or more nucleotide sequences encoding the one or more guide RNAs (gRNAs);
wherein said nucleotide sequences encoding the one or more guide RNAs and encoding the fusion protein are each operably linked to a promoter heterologous to said nucleotide sequence;
and
wherein the one or more guide RNAs are capable of forming a complex with the fusion protein in order to direct said fusion protein to bind to said target DNA sequence and modify the target DNA molecule.

64. (canceled)

65. The system of claim 63, wherein the target DNA molecule is within a cell.

66.-79. (canceled)

80. The system of claim 63, wherein nucleotide sequences encoding the one or more guide RNAs and the nucleotide sequence encoding a fusion protein are located on one vector.

81.-83. (canceled)

84. A method for modifying a target DNA molecule comprising a target sequence comprising:
a) assembling an RGN-deaminase-USP ribonucleotide complex in vitro by combining:
i) one or more guide RNAs capable of hybridizing to the target DNA sequence; and
ii) the fusion protein of claim 19;
under conditions suitable for formation of the RGN-deaminase-USP ribonucleotide complex; and
b) contacting said target DNA molecule or a cell comprising said target DNA molecule with the in vitro-assembled RGN-deaminase-USP ribonucleotide complex;
wherein the one or more guide RNAs hybridize to the target DNA sequence, thereby directing said fusion protein to bind to said target DNA sequence and modification of the target DNA molecule occurs.

85. (canceled)

86. The method of claim 84, wherein said modified target DNA molecule comprises a C>T mutation of at least one nucleotide within the target DNA sequence.

87.-96. (canceled)

97. The method of claim 84, wherein the target DNA molecule is within a cell.

98.-102. (canceled)

103. The method of claim 97, further comprising selecting a cell comprising said modified DNA molecule.

104.-111. (canceled)

112. A method for producing a genetically modified cell with a correction in a causal mutation for a genetically inherited disease, the method comprising introducing into a cell the system of claim 63,
whereby the fusion protein and the one or more gRNAs of the system target to the genomic location of the causal mutation and modify the genomic sequence to remove the causal mutation.

113.-114. (canceled)

115. The method of claim 112, wherein the genome modification comprises introducing a C>T mutation of at least one nucleotide within the target DNA sequence.

116.-119. (canceled)

120. A composition comprising:
a) a fusion protein comprising: (i) a DNA-binding polypeptide; and (ii) a deaminase; or a nucleic acid molecule encoding the fusion protein; and
b) a uracil stabilizing polypeptide (USP) having at least 80% sequence identity to any one of SEQ ID NOs: 1-16; or a nucleic acid molecule encoding the USP.

121. The composition of claim 120, wherein the fusion protein further comprises a uracil stabilizing polypeptide (USP) having at least 80% sequence identity to any one of SEQ ID NOs: 1-16.

122.-138. (canceled)

139. The nucleic acid molecule of claim 7, wherein the polynucleotide sequence comprises a nucleotide sequence having at least 80% sequence identity to the nucleotide sequence set forth as SEQ ID NO: 50.

140. The fusion protein of claim 23, wherein said RGN nickase has an amino acid sequence having at least 80% sequence identity to the amino acid sequence set forth as SEQ ID NO: 41, said deaminase has an amino acid sequence having at least 80% sequence identity to the amino acid sequence set forth as SEQ ID NO: 47, and said USP has an amino acid sequence having at least 80% sequence identity to the amino acid sequence set forth as SEQ ID NO: 1.

141. The fusion protein of claim 23, wherein said RGN nickase has an amino acid sequence having at least 80% sequence identity to the amino acid sequence set forth as SEQ ID NO: 41, said deaminase has an amino acid sequence having at least 80% sequence identity to the amino acid sequence set forth as SEQ ID NO: 77, and said USP has an amino acid sequence having at least 80% sequence identity to the amino acid sequence set forth as SEQ ID NO: 1.
